(19) United States
(12) Patent Application Publication
(10) Pub. No.: US 2011/0294217 A1
(43) Pub. Date: Dec. 1, 2011

(54) DNA NICKING ENZYME FROM A HOMING ENDONUCLEASE THAT STIMULATES SITE-SPECIFIC GENE CONVERSION

(75) Inventors: Audrey McConnell-Smith, San Diego, CA (US); Barry L. Stoddard, Bellevue, WA (US)

(73) Assignee: FRED HUTCHINSON CANCER RESEARCH CENTER, Seattle, WA (US)

(21) Appl. No.: 13/201,442

(22) PCT Filed: Feb. 12, 2010
(86) PCT No.: PCT/US2010/024153

§ 371 Date: Aug. 12, 2011

Related U.S. Application Data
(60) Provisional application No. 61/152,209, filed on Feb. 12, 2009.

(51) Int. Cl. C12N 9/16; C12N 15/63; C07H 21/04

(52) U.S. Cl. 435/440; 435/196; 536/23.2; 435/320.1

(57) ABSTRACT

An engineered highly specific DNA-cleavage enzyme delivers a site-specific nick in a double stranded DNA, to cleave one DNA strand within its target site while leaving the opposing DNA strand intact. The engineered enzyme provides the ability to induce a gene conversion event in a mammalian cell. An engineered sequence-specific nickase derived from a LAGLIDADG homing endonuclease is altered by a single amino acid residue, wherein the amino acid residue is involved in the polarization of solvent molecules and acid-base catalysis in the active site without affecting direct contacts between the enzyme and either the bound DNA or bound metal ions. Engineered, site-specific nickase variants, such as of I-AniI and other homing endonucleases, are particularly useful in targeted genome engineering as well as therapeutic, targeted gene repair.

### CROSS-REFERENCES TO RELATED APPLICATIONS

This application is a U.S. National Stage under 37 C.F.R. §371 based on International Application No. PCT/US2010/024153, filed Feb. 12, 2010, which claims priority to U.S. Provisional Application No. 61/152,209, filed Feb. 12, 2009, all of which are incorporated herein by reference.

### STATEMENT AS TO RIGHTS TO INVENTIONS MADE UNDER FEDERALLY SPONSORED RESEARCH OR DEVELOPMENT

The present invention was developed in part with government support under grant numbers R01 GM49857 and RL1 CA833133 awarded by the National Institutes of Health. The Government has certain rights in this invention.

### STATEMENT REGARDING SEQUENCE LISTING

The sequence listing associated with this application is provided in text format in lieu of a paper copy and is hereby incorporated by reference into the specification. The name of the text file containing the sequence listing is 37361_SEQ_FINAL_{—}20110720.txt. The name text file is 4/35 KB; was created on Jul. 20, 2011; and is being submitted via EFS-web with the filing of the specification.

### BACKGROUND OF THE INVENTION

Homing endonucleases generate sequence-specific DNA double-strand breaks (DSBs) that enable their genes, usually in concert with surrounding intron or intein sequences, to invade homologous host alleles that lack the intervening sequence (reviewed in Stoddard, Quart. Rev. Biophys. 38:49-55, 2005). Repair of the break by homologous recombination results in genetic transmission and persistence of these mobile elements (Dujon, Cell 20:185-197, 1980; Dujon, Gene 82:91-114, 1989; Belfort et al., J. Bacter. 177:3897-3903, 1995; Pleissis et al., Genetics, 130:451-460, 1992; Choulika et al., Mol. Cell Biol. 15:1968-1973, 1995). Homing endonucleases are promising candidates for gene modification reagents, as they recognize a broad range of long DNA target sites (14 to 40 basepairs) with great sequence specificity (reviewed in Chevalier et al., in Homing Endonucleases and Inteins, eds. Belfort et al., Springer Verlag, Vol. 16, pages 34-47, 2005; Paques, Curr. Gene Ther. 7:49-66, 2007). Members of one of the five known homing endonuclease families, the LAGLIDADG proteins, are especially promising as they exhibit the highest specificity, cleaving as few as 1 in 10⁸ to 10⁹ random DNA sequences (Gimble et al., J. Mol. Biol. 334:993-1008, 2003; Scalley et al., J. Mol. Biol. 372:1305-1319, 2007).

LAGLIDADG endonucleases contain two similar core folds of mixed α/β topology, with their namesake sequence motifs forming two α-helices that are packed together at the domain or subunit interface, where they each contribute a catalytic residue to an active site. Enzymes containing a single LAGLIDADG (SEQ ID NO: 1) motif per protein chain form homodimers that recognize palindromic and pseudo-palindromic DNA target sites, while those containing two motifs form asymmetric monomers that recognize correspondingly asymmetric DNA target sites.

To use LAGLIDADG endonucleases as gene-targeting reagents, particularly for therapeutic gene correction, it is essential that endonuclease-induced breaks are conservatively repaired. Naturally occurring LAGLIDADG enzymes create DNA DSBs which can be repaired by either homologous recombination or non-homologous end joining (NHEJ) (Wyamn, Ann. Rev. Genet. 40:363-383, 2006; Sung, Nat. Rev. Mol. Cell Biol. 7:739-750, 2006; Paques, Microbiol. Mol. Biol. Rev. 63:349-404, 1999; Brugmans et al., Mutat. Res. 614:95-108, 2007). Homologous recombination uses a homologous donor sequence as a template to conservatively repair the damaged site, while NHEJ directly rejoins the two free DNA ends. NHEJ usually results in sequence loss at the repair junction (Paques, Microbiol. Mol. Biol. Rev. 63:349-404, 1999; Lee et al., Cell 117:171-184, 2004), and can also promote chromosome translocations at DSBs, leading to genomic instability. Several homing endonucleases have been shown to cause such genomic instability as a result of NHEJ-mediated break repair (Weinstock et al., DNA Repair (Amst), 5:1065-1074, 2006; Rouet et al., Proc. Natl. Acad. Sci. USA 91:6064-6068, 1994; Monnat et al., Biochem. Biophys. Res. Commun. 255:88-93, 1999; Guirouilh-Barbat et al., Mol. Cell. 14:611-623, 2004; Allen et al., DNA Repair (Amst), 2:1147-1156, 2003).

An enzyme that created a DNA nick rather than a DSB might stimulate homologous recombination while reducing genomic instability associated with DSBs. While DSBs have an important role as initiators of homologous recombination (Szostak et al., Cell 33:25-35, 1983), several models for recombination readily accommodate initiation by nicks (Holliday, Genet. Res., 5:282-304, 1964; Radding, Ann. Rev. Genet. 16:405-437, 1982; Meselson et al., Proc. Natl. Acad. Sci. 72:358-361, 1975; Strathern et al., Genetics 127:61-73, 1991). Some members of the HNH family of homing endonuclease (such as the phage-derived enzyme I-HmuI) cut one strand of their DNA substrate and promote efficient intron homing (Landthaler et al., J. Mol. Biol. 358:1137-1151, 2006). The ability of a DNA nick to stimulate homologous recombination in mammalian cells has also been established by analysis of derivatives of the RAG proteins, which cleave DNA to promote V(D)J recombination at the immunoglobulin genes (Lee et al., Cell 117:171-184, 2004).

Naturally occurring dimeric restriction enzymes have been engineered to nick DNA at their short recognition sequences by inactivating or replacing one of the two subunits (Zhu et al., J. Mol. Biol. 337: 573-583, 2004; Xu et al., Nucl. Acids Res. 35:4608-4618, 2007; Samuelson et al., Nucl. Acids Res. 32:3661-3671, 2004; Heiter et al., J. Mol. Biol. 348:631-640, 2005). A strategy can be applied to convert a monomeric LAGLIDADG endonuclease to a nickase, by inactivating one of the two endonuclease active sites. These enzymes appear to employ a canonical 2-metal-ion mechanism of phosphoryl hydrolysis (FIG. 1A) (Chevalier et al., Nat. Struc. Biol. 8:312-316, 2001; Chevalier et al., Biochemistry 43:4015-4026, 2004; Moure et al., Nucl. Acids Res. 36:3287-3296, 2008). A conserved acidic residue from the carboxyl-terminus of each LAGLIDADG (SEQ ID NO: 1) motif coordinates bound metal ions in each of the two active sites, and more peripheral side chains participate in proton transfer and transition state stabilization. Mutation of these latter residues has been shown to abrogate DNA cleavage by the homodimeric endonuclease I-CreI (Chevalier et al., Biochemistry 43:14015-14026, 2004), and has produced nicking variants of the monomeric LAGLIDADG homing endonuclease I-SceI. In that study, mutation of either of two active site lysine residues produced a DNA nicking enzyme with significant sequence- and strand-specificity (Niu et al., J. Mol. Biol. 382:188-202, 2008).

The present disclosure describes the engineering of a sequence-specific nickase from a monomeric endonuclease that is capable of stimulating homologous recombination. In particular, the engineering of the monomeric endonuclease I-AniI to form a sequence-specific ‘nickase’ is described. In addition, it is demonstrated that the engineered nickase has the ability to promote targeted gene correction by homologous recombination in human cells. Comparisons of the engineered nickase and the parental ‘cleavase’ demonstrate that the two enzymes display similar solution behaviors, metal and pH dependence, DNA target site affinities and DNA sequence specificity. In addition, the nickase active site mutation can be successfully combined with mutations in the protein scaffold that increase physiological activity. Specifically the engineered I-AniI nickase stimulates gene conversion in human cells both in cis and in trans. As such, an engineered nickase from a LAGLIDADG homing endonuclease (LHE) provides a useful reagent for comparisons of homologous recombination, gene conversion and mutagenesis stimulated by single-versus double-strand breaks, and is valuable for a variety of genome engineering applications.

### BRIEF SUMMARY OF THE INVENTION

Provided herein is an engineered highly specific DNA-cleavage enzyme that can deliver a site-specific nick in a double stranded DNA. In particular, the engineered enzyme cleaves one DNA strand within its target site while leaving the opposing DNA strand intact. The engineered enzyme provides a construct that can induce a gene conversion event in a mammalian cell. As such, the present disclosure provides a general construct that can uncouple and inactivate an individual active site of a site specific DNA-cleavage enzyme by altering as little as a single amino acid. In a particular embodiment, the present disclosure provides an engineered sequence-specific nickase derived from a LAGLIDADG homing endonuclease by altering a single amino acid residue, wherein the amino acid residue is involved in the polarization of solvent molecules and acid-base catalysis in the active site without affecting direct contacts between the enzyme and either the bound DNA or bound metal ions. In a specific embodiment described herein a basic lysine residue at position 227 of I-AniI is substituted by a non-functional amino acid residue, such as methionine, to prevent activation of a water nucleophile in one endonuclease active site.

### BRIEF DESCRIPTION OF THE DRAWINGS

FIGS. 1A and 1B depict target sites and variant enzyme scaffolds for the endonuclease I-AniI. FIG. 1A. The wild-type I-AniI target site corresponds to a 19 base pair coding sequence in the mitochondrial cytochrome b oxidase gene in the host organism Aspergillus nidulans. The Lib4 target site was identified in an in vitro screen for cleavable target site variants (Scalley-Kim et al., J. Mol. Biol. 372:1305-1319, 2007). This sequence contains two base pair substitutions, highlighted, that increase binding affinity and cleavage by native I-AniI by approximately 5-fold. FIG. 1B. The “Y2” endonuclease scaffold of I-AniI (dark gray) differs from the native scaffold (light grey) at two residues, where F13Y and S111Y substitutions (see arrows) increase DNA binding affinity and improve catalytic activity at physiological temperatures (30 to 37° C.). Native I-AniI binds its target site with a dissociation constant (K_{D}) of approximately 90 nM and exhibits a temperature optima of about 55° C., ‘Y2’ I-AniI binds native target site DNA with a dissociation constant (K_{D}) of approximately 10 nM and a temperature optima of about 35° C. The Y2 construct was identified in an in vitro screen for enzyme variants that exhibit improved cleavage of native I-AniI target site DNA at 30° C.

FIGS. 2A through 2C depict the point mutations to generate an I-AniI nickase. FIG. 2A provides a ribbon diagram of wild-type I-AniI bound to its DNA target site. Positions Q171 and K227, in the periphery of the right active site that cleaves the top DNA strand, are indicated. FIG. 2B demonstrates the cleavage of a plasmid substrate containing the hypercleavable Lib4 I-AniI target sequence by wild-type, Q171K, K227M, and Q171K+K227M I-AniI variants at 10, 100 and 1000 nM enzyme. Digestion with I-HmuI provides a nicked substrate control, uncut plasmid and EcoRI-linearized plasmid are at right. The box indicates the K227M I-AniI variant as the strongest nickase. FIG. 2C depicts the nicking of the 313 bp end-labeled duplex substrate to generate predicted radiolabeled fragments of 254 and 63 nucleotides is diagrammed (left). Right, assays of cleavage and nicking by wild-type I-AniI, I-AniI nickase, and Y2 I-AniI nickase.

FIG. 3 demonstrates the DNA nicking and cleavage activity of Y2 I-AniI nickase. Top panel: Digests of supercoiled (sc) substrate plasmid with increasing concentrations of I-AniI Y2 nickase. Digests were for 2 hrs using 10 nM DNA plasmid substrate and I-AniI Y2 nickase protein ranging from 1 to 100 nM under digest condition described in the Examples. Bottom panel: quantitation of gel data shown in the top panel. Longer digests at 1 to 10 nM enzyme (≦1:1 molar ratio) did not generate detectable linearized product.

FIG. 4A: pH profile. Supercoiled substrate plasmid was digested with 1 μM WT or K227M I-AniI at the specified pH for 2 hr. FIG. 4B: Percent cleavage or nicking vs. pH for both enzymes. Gel bands were quantified with ImageJ®. Intensities of nicked or linearized plasmid were compared to that of supercoiled uncut plasmid in each lane to determine the percent cleavage. FIG. 4C: Metal-dependence profile. Supercoiled substrate plasmid was digested as in FIG. 4A at the specified MgCl₂ concentration. FIG. 4D: Percentage cleavage or nicking vs. magnesium concentration for WT and nicking I-AniI. Quantification as in FIG. 4B. FIG. 4E: Relative binding of double strand DNA (dsDNA) substrate by I-AniI K227M nickase, wild-type I-AniI cleavase, and a catalytically inactive I-AniI construct (harboring the double mutation K227M/Q171K) as measured by isothermal titration calorimetry. K_{D} values are averages of three independent experiments. Though the estimated molar ratios for binding for experiments shown in these panels are slightly below 1:1, the average of multiple runs in each case indicates a 1:1 binding stoichiometry of protein to DNA duplex.

FIG. 5A depicts a time course of DNA digestion by native I-AniI. Supercoiled substrate plasmid was digested with 1 μM WT I-AniI, and samples removed at 10 minute intervals over 2 hr. Complete linearization was achieved by 80 minutes. FIG. 5B depicts a time course of DNA digestion by I-AniI nickase, again using 1 μM enzyme concentration. Samples were removed at 0, 0.5, 1, 2, 3, 4, 7.5, 10, 15, 20, 25, and 30 minutes, and then at 10 minute intervals up to 90 minutes. The nicking reaction was more rapid than the wild-type cleavage reaction: complete nicking was achieved by approximately 50 minutes. FIG. 5C provides a plot of reaction time versus percent cleavage or nicking. Curves fit with program Prism5®: nicking t_{1/2}=7.6 minutes, cleavage t_{1/2}=59.6 minutes.

FIG. 6 provides a specificity profile of I-AniI nickase, determined by measuring the relative nickability of all individual single base pair variants of the wild-type I-AniI target site. Results are shown as fraction of nicked plasmid substrate recovered (0.5=50% nicked) versus base pair variant for each target site position. The bold line illustrates the relative cleavage of the wild-type target site, measured as a control experiment during each set of digests. Examples of digests are shown in FIG. 7.

FIG. 7 provides data demonstrating the ability of I-AniI nickase to cleave target sites with one base pair substitutions. A target site matrix consisting of 60 separate base substitutions was used to determine the ability of I-AniI nickase to cleave mutant target sites. A single preparation of I-AniI nickase protein was used to generate the cleavage results shown over four days (the day number is show at the left of each panel). On each day, a standard series of digests using a native I-AniI target site was performed to control for loss or change in specific activity; none was detected. Rows contain control native site digests in the far left column (WT), followed by pairs of lanes that represent a no-enzyme control (left) and digest products (right) for a specific mutant target site. A marker DNA ladder (most with an additional DNA MW control corresponding to linearized substrate plasmid) is shown in the far right column. Cleavage experiments were performed three times, and the averaged extent of cleavage for each substrate was used to construct the relative cleavage plot shown in FIG. 6.

FIG. 8 describes the ability of I-AniI cleavase and I-AniI nickase, both in the presence of the “Y2” scaffold, to induce homologous recombination in transfected human cells.

FIGS. 8A and 8B depict the reporter constructs. The assay measured recombination between two nonfunctional GFP genes or gene fragments (dark grey) to create a functional GFP gene. The acceptor GFP gene was interrupted by an I-AniI site and a stop codon (black box with the white x); the homologous donor (GFPi) was truncated on both 5′- and 3′-end. To assay recombination in cis, both donor and acceptor were on a single non-replicating plasmid. To assay recombination in trans, the acceptor was integrated into the cellular chromosome and the correction donor repair template was provided by transfection. In both assays, generation of either a double-strand break (DSB) or single-strand nick by I-AniI at the indicated site triggered a repair event in which the truncated copy of the GFP gene served as repair template leading to gene conversion and a functional GFP copy (GFP⁺, lower line). FIGS. 8C and 8D demonstrate changes in GFP expression in the presence of I-AniI variants indicated, representing recombination either in cis or in trans. Increase of GFP positive cells following expression of I-AniI cleavase “c” or I-AniI nickase “n.” FIG. 8C: recombination in cis; FIG. 8D: recombination in trans.

FIGS. 9A and 9B depict the plasmid-based gene conversion in cis in human cells. FIG. 9A: The DR-GFPAni reporter consists of two nonfunctional GFP genes (dark grey, top row). The upstream GFP gene is interrupted by an I-AniI site and a stop codon (Black box with the white X). The homologous sequence in the downstream copy is truncated on both 5′- and 3′-ends. Generation of a DSB (or nick) by I-AniI in the upstream GFP gene triggers a repair event in which the truncated copy of the GFP gene serves as a repair template leading to gene conversion and a functional GFP copy (GFP⁺, lower left). These events are detected and quantified by Fluorescent Activated Cell Sorting (FACS). Alternatively, the DSB (or nick) ends can be rejoined (GFP⁻, lower right). Accurate rejoining restores the I-AniI site, whereas inaccurate repair after end resection or addition generates an I-AniI resistant, GFP-mutant allele. FIG. 9B: Examples of flow output of 293T cells transfected with the DR-GFPAni reporter shown in FIG. 9A and either no or an I-AniI Y2 expression plasmid as described in the Examples. The two panels on the left show the background fluorescence in mock-transfected cells, or cells transfected with DR-GFPAni reporter plasmid. The two panels on the right show the FACS analysis of cells co-transfected with the pDR-GFPAni reporter plasmid and either an I-AniI Y2 cleavase (+I-AniI) or I-AniI Y2 nickase (+NI-AniI) expression vector. The percentage of GFP⁺ cells is shown in each panel. Transfection efficiency in all experiments was >90% as assessed by transfection of a control pEGFP-C1 coding plasmid (Clontech).

FIG. 10 depicts the expression levels of I-AniI variants transfected into 293T human cells. Western blot analysis was used to confirm and estimate the level of expression of I-AniI proteins in vivo. Cleavage-competent (c=cleavase) or I-AniI site-nicking (n=nickase) variants of native or Y2 variant I-AniI were expressed by transient transfection of expression vectors into human 293T cells. Cellular extracts were prepared from transfected cell used for GFP+ recombination analyses shown in FIG. 9.

FIG. 11 depicts amounts of homologous recombination occurring in transfected cells containing an integrated inactive lacZ target, by double-strand break-inducing enzyme (DSB AniY2) or nicking enzyme (AniY2), at two different target lacZ sites, one in which the 19 bp I-AniI recognition site replaced a 19 bp region in the lacZ gene, and the other in which the I-AniI site was inserted into the same location.

### DETAILED DESCRIPTION OF THE INVENTION

Generally, the nomenclature used herein and many of the laboratory procedures in regard to cell culture, molecular genetics and nucleic acid chemistry, which are described below, are those well known and commonly employed in the art. (See generally Sambrook et al., Molecular Cloning: A Laboratory Manual, 3d Ed., Cold Spring Harbor Laboratory Press, New York (2001), which is incorporated by reference herein). Standard techniques are used for recombinant nucleic acid methods, site directed mutagenesis, preparation of biological samples, preparation of cDNA fragments, PCR, and the like. Generally enzymatic reactions and any purification and separation steps using a commercially prepared product are performed according to the manufacturers' specifications.

LAGLIDADG homing endonucleases are highly site-specific DNA-cleaving enzymes capable of inducing gene conversion by generating double-strand breaks that are repaired via homologous recombination. These enzymes are potentially valuable tools for targeted gene correction and genome engineering. The present disclosure describes a method for the construction of a variant of a highly specific DNA cleavage enzyme having a mutation in a single active site, wherein the variant enzyme nicks a DNA target sequence comprising the nucleotide sequence of the site specific DNA cleavage enzyme target site. In a particular embodiment, the method results in a variant of the I-AniI homing endonuclease that nicks its cognate target site (herein referred to as a “nickase”). In this embodiment the variant contains a mutation of a basic amino acid residue essential for proton transfer and solvent activation in one active site. The cleavage mechanism displayed by the remaining active site, and the DNA binding affinity and substrate specificity profile of the nickase, are similar to the native enzyme. I-AniI nickase stimulates targeted gene correction in human cells, in cis and in trans, at approximately one-quarter the efficiency of the native enzyme. The development of a gene-specific nicking enzyme facilitates comparative analyses of gene conversion, DNA repair efficiency and mutagenesis induced by single-versus double-strand breaks.

The site specific DNA cleavage enzyme that can be used to engineer a variant site specific DNA nickase enzyme of the present disclosure can include, for example, I-AniI, I-SceI, I-ChuI, I-CreI, I-CsmI, PI-TliI, PI-MtuI, I-CeuI, I-SceII, I-SceIII, HO, PI-CivI, PI-CtrI, PI-AaeI, PI-BsuI, PI-DhaI, PI-DraI, PI-MavI, PI-MchI, PI-MfuI, PI-MflI, PI-MgaI, PI-MgoI, PI-MniI, PI-MkaI, PI-MleI, PI-Mural, PI-MshI, PI-MsmI, PI-MthI, PI-MtuI, PI-MxeI, PI-NpuI, PI-PfuI, PI-RmaI, PI-SpbI, PI-SspI, PI-FacI, PI-MjaI, PI-PhoI, PI-TagI, PI-ThyI, PI-TkI, PI-TspI, and I-MsoI. Methods to convert those enzymes listed above that are natural homodimers (such as I-CreI, I-CeuI, I-MsoI) into single chain, monomeric enzymes are available to the skilled artisan (see for example, Li et al., Nucl. Acids Res. 37: 1650-1662, 2009). Active sites within the amino acid sequence of each one of the above examples are either known or determinable by methods known to the skilled artisan. The amino acid within an individual active site to be mutated is also known or can be determined, as are methods for site directed mutagenesis to change the selected amino acid residue. The amino acid substitution provides for an amino acid residue that fails to provide the requisite combination of size and pKₐ of a lysine sidechain for catalysis of a phosphotransfer reaction. Amino acid substitutions can include, for example, methionine (M), alanine (A), glutamine (Q), asparagine (N), leucine (L), and the like. In a particular embodiment, K227M, the lysine (K) residue at position 227 of I-AniI was changed to methionine (M).

In additional embodiments, the site specific DNA cleavage enzyme can be an enzyme that has an amino acid sequence that has been modified to change the site specificity from the wild-type target sequence, or that has been modified to improve binding site binding efficiency, cleavage efficiency, or some other characteristic of the enzyme. For example, in one embodiment a mutation of the site specific DNA cleavage enzyme I-AniI comprises a tyrosine (Y) substitution at positions 13 and 111 (the phenylalanine (F) at position 13 and the serine (S) at position 111 are changed to tyrosine (Y)) that increases binding affinity and cleavage by five-fold as compared with the wild-type I-AniI. In another embodiment, mutation in the DNA-binding surface of I-AniI, comprising the mutations G33R/V56I/R59S/A68R (the glycine (G) at position 33 was changed to arginine (R), the valine (V) at position 56 was changed to isoleucine (I), the arginine (R) at position 59 was changed to serine and the alanine (A) at position 68 was changed to arginine (R), alters the specificity of the enzyme toward different DNA base pairs at positions −4 and −5 of the enzyme's target site (from −4 G and −5 A to −4 C and −5 C). Additional mutations in the I-AniI enzyme scaffolding are also known to improve the solution behavior of the enzyme. The methods of the present disclosure can also be used with site specific DNA cleavage enzymes that have an enzyme scaffolding that has been modified in the same or a similar manner.

Provided herein are also vectors comprising the nucleic acid sequence that encodes the variant site specific DNA cleavage enzyme of the present disclosure. A nucleic acid encoding one or more engineered nickase or engineered nickase fusion protein can be cloned into a vector for transformation into prokaryotic or eukaryotic cells for replication and/or expression. Vectors can be prokaryotic vectors, e.g., plasmids, or shuttle vectors, insect vectors, or eukaryotic vectors. A nucleic acid encoding an engineered nickase as disclosed herein can also be cloned into an expression vector, for administration to a plant cell, animal cell, a mammalian cell or a human cell, fungal cell, bacterial cell, or protozoan cell.

To obtain expression of a cloned gene or nucleic acid, sequences encoding an engineered nickase or nickase fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression. A Laboratory Manual (1990); and Current Protocols in Molecular Biology (Ausubel et al., supra.) Bacterial expression systems for expressing the engineered nickase are available in, e.g., E. coli, Bacillus sp., and Salmonella (Palva et al., Gene 22:229-235, 1983). Kits for such expression systems are commercially available.

Eukaryotic expression systems for mammalian cells, yeast, and insect cells are well known by those of skill in the art and are also commercially available.

The promoter used to direct expression of an engineered nickase-encoding nucleic acid depends on the particular application. For example, a strong constitutive promoter is typically used for expression and purification of an engineered nickase. In contrast, when an engineered nickase is administered in vivo for gene regulation, either a constitutive or an inducible promoter is used, depending on the particular use of the engineered nickase. In addition, a promoter for administration of an engineered nickase can be a weak promoter, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen and Bujard, Proc. Nat'l. Acad. Sci. 89:5547, 1992; Oligino et al., Gene Ther. 5:491-496, 1998; Wang et al., Gene Ther. 4:432-441, 1997; Neering et al., Blood 88:1147-1155, 1996; and Rendahl et al., Nat. Biotechnol. 16:757-761, 1998). The MNDU3 promoter can also be used, and is preferentially active in CD34⁺ hematopoietic stem cells.

In addition to the promoter, the expression vector typically contains a transcription unit or expression cassette that contains all the additional elements required for the expression of the nucleic acid in a host cell, either prokaryotic or eukaryotic. A typical expression cassette thus contains a promoter operably linked, e.g., to a nucleic acid sequence encoding the engineered nickase, and signals required, e.g., for efficient polyadenylation of the transcript, transcriptional termination, ribosome binding sites, or translation termination. Additional elements of the cassette may include, e.g., enhancers, and heterologous splicing signals.

The particular expression vector used to transport the genetic information into the cell is selected with regard to the intended use of the engineered nickase, e.g., expression in plants, animals, bacteria, fungus, protozoa, and the like. Standard bacterial expression vectors include plasmids such as pBR322-based plasmids, pSKF, pET23D, pBluescript® based plasmids, and commercially available fusion expression systems such as GST and LacZ. An exemplary fusion protein is the maltose binding protein, “MBP.” Such fusion proteins are used for purification of the engineered nickase. Epitope tags can also be added to recombinant proteins to provide convenient methods of isolation, for monitoring expression, and for monitoring cellular and subcellular localization, e.g., a nuclear localization signal (NLS), an HA-tag, c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A⁺, pMT010/A⁺, pMAMneo-5, bacculovirus pDSVE, pCS, pEF, and any other vector allowing expression of proteins under the direction of the SV40 early promoter, SV40 late promoter, metallothionein promoter, murine mammary tumor virus promoter, Rous sarcoma virus promoter, polyhedrin promoter, elongation factor 1 promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with an engineered nickase encoding sequence under the direction of the polyhedrin promoter or other strong baculovirus promoters.

The elements that are typically included in an expression vector also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (Guide to Protein Purification, in Methods in Enzymology, vol. 182 (Deutscher, ed., 1990)). Transformation of eukaryotic and prokaryotic cells are performed according to standard techniques well known to the skilled artisan.

Any of the well known procedures for introducing foreign nucleotide sequences into host cells can be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

Nucleic acids encoding an engineered nickase as described herein and delivery to cells can use conventional viral and non-viral based gene transfer methods (e.g., mammalian cells) and target tissues. Such methods can also be used to administer nucleic acids encoding an engineered nickase to a cell in vitro. In certain embodiments, nucleic acids encoding an engineered nickase are administered for in vivo or ex vivo gene modification uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids encoding engineered nickases include electroporation, lipofection, microinjection, biolistics, virosomes, liposomes, immunoliposomes, polycation or lipid: nucleic acid conjugates, naked DNA, artificial virions, and agent-enhanced uptake of DNA. Sonoporation using, e.g., the Sonitron 2000 system (Rich-Mar) can also be used for delivery of nucleic acids.

Lipofection is described in e.g., U.S. Pat. No. 5,049,386, U.S. Pat. No. 4,946,787; and U.S. Pat. No. 4,897,355) and lipofection reagents are sold commercially (e.g., Transfectam® and Lipofectin™). Cationic and neutral lipids that are suitable for efficient receptor-recognition lipofection of polynucleotides include those of Feigne, WO1991/17424, WO1991/16024. Delivery can be to cells (ex vivo administration) or target tissues (in vivo administration). The preparation of lipid: nucleic acid complexes, including targeted liposomes such as immunolipid complexes, is well known to one of skill in the art

The use of RNA or DNA viral based systems for the delivery of nucleic acids encoding an engineered nickase takes advantage of highly evolved processes for targeting a virus to specific cells in the body and trafficking the viral payload to the nucleus. Viral vectors can be administered directly to patients or they can be used to treat cells in vitro and the modified cells are administered to patients. Conventional viral based systems for the delivery of an engineered nickase include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof.

In applications in which transient expression of an engineered nickase is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene modification procedures. Construction of recombinant AAV vectors are described in a number of publications, including U.S. Pat. No. 5,173,414; Tratschin et al., Mol. Cell. Biol. 5:3251-3260, 1985; Tratschin et al., Mol. Cell. Biol. 4:2072-2081, 1984; Hermonat and Muzyczka, Proc. Natl. Acad. Sci. 81:6466-6470, 1984; and Samulski et al., J. Virol. 63:03822-3828, 1989. Recombinant adeno-associated virus vectors (rAAV) provide an alternative gene delivery systems based on the defective and nonpathogenic parvovirus adeno-associated type 2 virus.

Replication-deficient recombinant adenoviral vectors (Ad) can be produced at high titer and readily infect a number of different cell types. Most adenovirus vectors are engineered such that a transgene replaces the AdE1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity.

Packaging cells are used to form virus particles that are capable of infecting a host cell. Such cells include 293 cells, which package adenovirus, and 2 cells or PA317 cells, which package retrovirus. Viral vectors used in gene modification are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are typically supplied in trans by the packaging cell line. For example, AAV vectors used in gene modification typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line can also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Gene modification vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene modification (e.g., via re-infusion of the transfected cells into the host organism) is well known to those of skill in the art. In a typical embodiment, cells are isolated from the subject organism, transfected with an engineered nickase nucleic acid (gene or cDNA), and re-infused back into the subject organism (e.g., a patient). Various cell types suitable for ex vivo transfection are well known to those of skill in the art (see, e.g., Freshney et al., Culture of Animal Cells, A Manual of Basic Technique (5th ed. 2005)) and the references cited therein for a discussion of how to isolate and culture cells from a patient).

Vectors (e.g., retroviruses, adenoviruses, liposomes, etc.) containing an engineered nickase nucleic acid can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available (see, e.g., Remington The Science and Practice of Pharmacy, 21^{st} ed., 2005).

DNA constructs may be introduced into the genome of a desired plant host by a variety of conventional techniques. For reviews of such techniques see, for example, Weissbach and Weissbach, Methods for Plant Molecular Biology (1988, Academic Press, N.Y.) Section VIII, pp. 421-463; and Grierson and Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment.

Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature and will not be described here.

Alternative gene transfer and transformation methods include, but are not limited to, protoplast transformation through calcium-, polyethylene glycol (PEG)- or electroporation-mediated uptake of naked DNA and electroporation of plant tissues. Additional methods for plant cell transformation include microinjection, silicon carbide mediated DNA uptake, and microprojectile bombardment.

The disclosed methods and compositions can be used to make genomic changes and/or to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is a well known technique to the skilled artisan. Regeneration can also be obtained from plant callus, explants, organs, pollens, embryos or parts thereof.

Nucleic acids introduced into a plant cell can be used to confer desired traits on essentially any plant. A wide variety of plants and plant cell systems can be engineered for the desired physiological and agronomic characteristics described herein using the nucleic acid constructs of the present disclosure and the various transformation methods mentioned above. Typically, target plants and plant cells for modification include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed (canola)) and plants used for experimental purposes (e.g., Arabidopsis).

One of skill in the art will recognize that after the expression cassette is stably incorporated in a transgenic plant and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, green fluorescent protein, luciferase, B or Cl genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, siRNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene activity can be used.

Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or western blotting). The transgene may be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

### Delivery Vehicles

An important factor in the administration of polypeptide compounds, such as an engineered nickase, and a vector encoding an engineered nickase, is ensuring that the polypeptide or vector construct has the ability to traverse the plasma membrane of a cell, or the membrane of an intra-cellular compartment such as the nucleus. Proteins and other compounds such as liposomes have been described and are known to the skilled artisan, which have the ability to translocate polypeptides such as an engineere nickase across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. Toxin molecules also have the ability to transport polypeptides across cell membranes. Often, such molecules (called “binary toxins”) are composed of at least two parts: a translocation/binding domain or polypeptide and a separate toxin domain or polypeptide. Typically, the translocation domain or polypeptide binds to a cellular receptor, and then the toxin is transported into the cell. Typically, the translocation sequence is provided as part of a fusion protein. Optionally, a linker can be used to link the engineered nickase and the translocation sequence. Any suitable linker can be used, e.g., a peptide linker.

The engineered nickase and constructs encoding the engineered nickases can also be introduced into an animal cell, preferably a mammalian cell, via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., an engineered nickase or vector encoding the nickase. The liposome fuses with the plasma membrane, thereby releasing the engineered nickase into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, the engineered nickase) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane. When liposomes are endocytosed by a target cell, for example, they become destabilized and release their contents.

Such liposomes typically comprise an engineered nickase and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes, and are well known in the art. Suitable methods include, for example, sonication, extrusion, high pressure/homogenization, microfluidization, detergent dialysis, calcium-induced fusion of small liposome vesicles and ether-fusion methods, all of which are known to those of skill in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been and methods for their construction and administration are well known to the skilled artisan. Standard methods for coupling targeting agents to liposomes can be used. These methods generally involve incorporation into liposomes of lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A.

The dose of an engineered nickase administered to a patient, or to a cell which will be introduced into a patient, in the context of the present disclosure, should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy and K_{d} of the particular engineered nickase employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

The maximum therapeutically effective dosage of an engineered nickase for approximately 99% binding to target sites is calculated to be in the range of less than about 1.5×10⁵ to 1.5×10⁶ copies of the specific engineered nickase molecule per cell. The appropriate dose of an expression vector encoding an engineered nickase can also be calculated by taking into account the average rate of engineered nickase expression from the promoter and the average rate of engineered nickase degradation in the cell. In certain embodiments, a weak promoter such as a wild-type or mutant HSV TK promoter is used, as described above. The dose of engineered nickase in micrograms is calculated by taking into account the molecular weight of the particular engineered nickase being employed.

In determining the effective amount of the nickase to be administered in the treatment or prophylaxis of disease, the physician evaluates circulating plasma levels of the nickase or nucleic acid encoding the engineered nickase, potential engineered nickase toxicities, progression of the disease, and the production of anti-nickase antibodies. Administration can be accomplished via single or divided doses.

Pharmaceutical compositions and administration of an engineered nickase and expression vectors encoding an engineered nickase can be administered directly to the patient for targeted single strand cleavage and/or recombination, and for therapeutic or prophylactic applications, for example, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, and the like. Examples of microorganisms that can be inhibited by nickase gene modification include pathogenic bacteria, e.g., Chlamydia, rickettsial bacteria, mycobacteria, staphylococci, streptococci, pneumococci, meningococci and gonococci, Klebsiella, Proteus, Serratia, Pseudomonas, Legionella, Diphtheria, Salmonella, bacilli, Cholera, tetanus, botulism, anthrax, plague, leptospirosis, and Lyme disease bacteria; infectious fungus, e.g., Aspergillus, Candida species; protozoa such as sporozoa (e.g., Plasmodia), rhizopods (e.g., Entamoeba) and flagellates (Trypanosoma, Leishmania, Trichomonas, Giardia, and the like); viral diseases, e.g., hepatitis (A, B, or C), herpes virus (e.g., VZV, HSV-1, HSV-6, HSV-11, CMV, and EBV), HIV, Ebola, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, poliovirus, rabies virus, and arboviral encephalitis virus, and the like.

Administration of therapeutically effective amounts is by any of the routes normally used for introducing an engineered nickase or an expression vector encoding an engineered nickase of the invention into ultimate contact with the tissue or cell type to be treated. The engineered nickase is administered in any suitable manner, preferably with a pharmaceutically acceptable carrier. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences, 18th ed., 1990).

The engineered nickase, alone or in combination with other suitable components, can be made into an aerosol formulation (i.e., “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally.

The formulations of compounds can be presented in unit-dose or multi-dose sealed containers, such as ampules and vials. Injection solutions and suspensions can be prepared from sterile powders, granules, and tablets of the kind previously described.

### Applications

The disclosed methods and engineered nickase compositions for targeted cleaving one strand of a polynucleotide sequence can be used to induce mutations in a genomic sequence, e.g., by nicking a single strand in the region of its genomic target sequence and initiating enzymantic events and subsequent mechanisms in the cell that lead to gene conversion and repair shifted to conservative, templated recombination pathways. The same methods can also be used to replace a wild-type sequence with a mutant sequence, or to convert one allele to a different allele.

Targeted single strand cleavage (nicking) of infecting or integrated viral genomes can be used to treat viral infections in a host. Additionally, targeted single strand cleavage of genes encoding receptors for viruses can be used to block expression of such receptors, thereby preventing viral infection and/or viral spread in a host organism. Targeted mutagenesis of genes encoding viral receptors can be used to render the receptors unable to bind to virus, thereby preventing new infection and blocking the spread of existing infections. Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, and the like); Caliciviridae; Togaviridae (e.g., rubella virus, dengue virus, and the like); Flaviviridae; Coronaviridae; Reoviridae; Birnaviridae; Rhabodoviridae (e.g., rabies virus, and the like); Filoviridae; Paramyxoviridae (e.g., mumps virus, measles virus, respiratory syncytial virus, and the like); Orthomyxoviridae (e.g., influenza virus types A, B and C, and the like); Bunyaviridae; Arenaviridae; Retroviradae; lentiviruses (e.g., HTLV-I; HTLV-II; HIV-1, HIV-II); simian immunodeficiency virus (SIV), human papillomavirus (HPV), influenza virus and the tick-borne encephalitis viruses. See, e.g., Fundamental Virology, 2nd Edition (Fields Knipe, eds. 1991), for a description of these and other viruses.

In similar fashion, the genome of an infecting bacterium can be mutagenized by targeted single strand DNA cleavage followed by templated recombination, to block or ameliorate bacterial infections. The disclosed methods for targeted homologous recombination can be used to replace any genomic sequence with a homologous, non-identical sequence. For example, a mutant genomic sequence can be replaced by its wild-type counterpart, thereby providing methods for treatment of, e.g., genetic disease, inherited disorders, cancer, and autoimmune disease. In like fashion, one allele of a gene can be modified using the methods of targeted recombination disclosed herein.

Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, Canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GMI), hemochromatosis, the hemoglobin C mutation in the 6 codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome (XLP, OMIM No. 308240).

Additional exemplary diseases that can be treated by targeted single DNA strand cleavage and/or targeted templated homologous recombination of the invention include acquired immunodeficiencies, lysosomal storage diseases (e.g., Fabry disease), mucopolysaccahidosis (e.g., Hunter's disease), hemoglobinopathies and hemophilias. In certain cases, alteration of a genomic sequence in a pluripotent cell (e.g., a hematopoietic stem cell) is desired. Methods for mobilization, enrichment and culture of hematopoietic stem cells are known in the art. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or its control sequences with sequences that support a lower, non-pathologic level of expression. Any pathology dependent upon a particular genomic sequence, in any fashion, can be corrected or alleviated using the methods and compositions disclosed herein.

Targeted single DNA strand cleavage and targeted template recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

The engineered nickase compositions and methods described herein also allow for novel approaches and systems to address immune reactions of a host to, for example, allogeneic grafts. In particular, a major problem faced when allogeneic stem cells (or any type of allogeneic cell) are grafted into a host recipient is the high risk of rejection by the host's immune system, primarily mediated through recognition of the Major Histocompatibility Complex (MHC) on the surface of the engrafted cells. The MHC comprises the HLA class I protein (s) that function as heterodimers that are comprised of 3 common subunits and a variable subunit. It has been demonstrated that tissue grafts derived from stem cells that are devoid of HLA escape the host's immune response. Using the engineered nickase compositions and methods described herein, genes encoding HLA proteins involved in graft rejection can be cleaved, mutagenized or altered by templated recombination, in either their coding or regulatory sequences, so that their expression is blocked or they express a non-functional product. For example, by inactivating the gene encoding the common β subunit gene (β₂ microglobulin) using an engineered nickase as described herein, HLA class I can be removed from the cells to rapidly and reliably generate HLA class I null stem cells from any donor, thereby reducing the need for closely matched donor/recipient MHC haplotypes during stem cell grafting.

Inactivation of a gene (e.g., the β₂ microglobulin or other gene) can be achieved, for example, by a single cleavage event, by cleavage followed by templated recombination, by targeted recombination of a mis sense or nonsense codon into the coding region, or by targeted recombination of an irrelevant sequence (i.e., a “stuffer” sequence) into the gene or its regulatory region, so as to disrupt the gene or regulatory region.

### EXAMPLES

The present example provides for the production, expression and purification of a LAGLIDADG homing endonuclease engineered to nick its cognate DNA target site.

### Protein Expression, Purification and Mutagenesis.

Expression and purification protocols used herein were similar to those published previously (Bolduc et al., Genes Dev. 17:2875-2888, 2003). All I-AniI scaffolds included mutations that replace the phenylalanine at position 80 and the leucine at position 233 with lysine (F80K and L233K, respectively), shown to improve solution behavior of the enzyme (Scalley-Kim et al., J. Mol. Biol. 372:1305-1319, 2007). The “Y2” variant contains two additional mutations that replace the phenylalanine at position 13 and the serine at position 111 with tyrosine (F13Y and S111Y, respectively), which enhance both DNA binding affinity and cleavage efficiency at physiological temperatures. I-AniI point mutants were generated by site directed mutagenesis using QuickChange® XL (Stratagene). Oligonucleotides (Operon) used to generate K227M were 5′-CAAAATGCGCCTGTCAAATTATTAGGGAATATGAAATTACA ATATAAATTATGGTT AAAAC-3′(SEQ ID NO: 2) and its complement, and to generate Q171K were 5′-CGCTAGTT TTGATATTGCTAAACGCGATGG GGATATTCTG-3′(SEQ ID NO: 3) and its complement. Mutations were verified by direct sequencing of expression constructs.

### Cleavage and Nicking Assays.

Cleavage was carried out as previously described (Scalley-Kim et al., J. Mol. Biol. 372:1305-1319, 2007) in reactions containing 10 nM BlueScript® plasmid substrate (pBS) containing the I-AniI Lib4 target site and 1 μM I-AniI, unless otherwise specified; this enzyme concentration is well over K_{D} for the interaction between I-AniI and its DNA target site. Most reactions were carried out in 50 mM Tris (pH 7.5), 100 mM NaCl, 10 mM MgCl₂, and 1 mM DTT, and incubated at 37° C. for 2 hr. For pH profiles, Tris was substituted by: sodium citrate (pH 5.0, 5.5), Bis-Tris (pH 6.0, 6.5), Tris (pH 7.0-9.0), and CAPS (pH 9.5-10.5). Reactions were terminated by the addition of an equal volume of 2× stop buffer (2% SDS, 100 mM EDTA, 20% glycerol, and 0.2% bromophenol blue). Control digests with EcoRI and the nickase I-HmuI were performed under the same conditions.

To generate the 313 bp end-labeled duplex cleavage substrate, an I-AniI Lib4 target site was amplified from pBS using primers SP149 (5′-CGTAATACGACTCACTATAGG-3′ (SEQ ID NO: 4)) and SP150 (5′-CGCAATTAATGTGAGTTAGCT-3′ (SEQ ID NO: 5)), products purified on Illustra ProbeQuant® G50 columns (GE Healthcare), and 5′ end-labeled with γ³²P-ATP (Perkin Elmer) and T4 polynucleotide kinase (NEB) according to the manufacturer's protocol. Following cleavage in 10 μl reaction volume, 2 μl of 5× stop solution (0.1 M TrisHCl pH 7.5, 0.25 M EDTA, 5% SDS) was added and samples denatured at 95° C. for 5 min with deionized formamide, 0.1% xylene cyanol, and 0.1% bromophenol blue, quick-chilled, then resolved by electrophoresis on a 6% polyacrylamide denaturing gel. Gels were dried and analyzed by Phosphorimager®.

### Nicking Site-Specificity.

A plasmid substrate containing the wild-type (WT) I-AniI target site was generated by cloning a synthetic double-stranded 40 bp DNA cassette containing the target site sequence (Scalley-Kim et al., J. Mol. Biol. 372:1305-1319, 2007) into the EcoRI and XhoI sites of the pBlueScript plasmid vector multicloning region. A matrix of 60 separate target site variants was generated using site-directed mutagenesis (QuikChange®, Stratagene), and verified by chemical sequencing. Each variant in the matrix contained a single base pair substitution at one position; all three possible substitutions were made at each of 20 positions. Reactions contained 5 nM DNA substrate and 20 nM I-AniI nickase; and the entire analysis was carried out with a single enzyme preparation to ensure uniformity. For each substrate plasmid, a “no enzyme” control incubation was conducted in parallel to quantitate non-enzymatic hydrolysis.

### In Vivo Recombination Assays.

Reporter plasmids pDR-GFPAni and pDR-GFPLib4 were constructed by modifying the original pDR-GFP recombination reporter (Pierce et al., Genes Dev. 13:2633-2638, 1999). The I-SceI recognition site in the 5′ SceGFP cassette was replaced by a multiple cloning site containing SacI, KpnI, and XhoI cleavage sites, generated by annealing oligonucleotides Usrf, 5′-GAGCTCGGTACCCTCGAGGCCGGACACGCTGAACTTG-3′ (SEQ ID NO: 6) and Usrr, 5′-CTCGAGGGTACCGAGCTCACCTACGGCAAGCTGACC-3′ (SEQ ID NO: 7), to generate pusrDR-GFP. This plasmid was then cleaved with SacI and XhoI, and the following annealed duplexes were inserted to generate pDR-GFPAni or pDR-GFPLib4, respectively: Anif, 5′-TCGATGAGGAGGTTTCTCTGTAAAGCT-3′ (SEQ ID NO: 8) and Anir, 5′-TTACAGAG AAACCTCCTCA-3′ (SEQ ID NO: 9); and Lib4f, 5′-TCGATGAGGAGGTTACTCTGTTAT AACAGCTGAGCT-3′ (SEQ ID NO: 10) and Lib4r, 5′-CAGCTGTTATAACAGAGTAACCTC CTCA-3′ (SEQ ID NO: 11).

Plasmid pZF-GFPAni was constructed from pDR-GFP by removal of the downstream, truncated GFP gene by partial HindIII digestion and religation, insertion of the MboI fragment containing an approximately 4 kb poly-lacO array from plasmid φV-lacO-His (Cummings et al., PLoS Biol. 5:2145-2155, 2007) into the NotI site downstream of the 5′ SceGFP cassette, and insertion of a duplex containing the Lib4 site, generated by annealing oligonucleotides AniCut.F1,5′-GGTGAGGAGGTTACTCTGTTATAGGGATAA-3′ (SEQ ID NO: 12) and AniCut.R1, 5′-CCCTATAACAGAGTAACCTCCTCACCTTAT-3′ (SEQ ID NO: 13) into the I-SceI site. The downstream, truncated GFP gene was excised by HindIII digestion from pDR-GFP and cloned into the HindIII site of pBS-SK⁺ to generate the truncated GFP donor plasmid, iGFP.

The I-AniI coding plasmids, pCSOMpEFHA-2ndGenNLS-HyperAniKWPRE and pRRLSIN.cPPT.hPGK.HA.2ndGenNLS.reoAniY2 were obtained from M. Certo and A. Scharenberg (Seattle Children's Hospital, Seattle, Wash.). In these, a pEF1α promoter drives I-AniI ORFs that include an N-terminal HA-tag and NLS, and the ORF contains F80K and L232K substitutions as well as a silent mutation (G25G) that was necessary to abolish a cryptic splice site. The latter Y2 scaffold also contains the F13Y and S111Y mutations. The I-AniI nickase ORF contains an additional K227M substitution, constructed by site directed mutagenesis (QuickChange®, Stratagene) using oligonucleotide 5′-CCTGTCAAATTGTTAGGCAACAtGA AACTGCAATACAAGTTGTGG-3′ (SEQ ID NO: 14) and its complement.

For transfections, human 293T cells were grown in Dulbecco-modified Eagle's medium (Cellgro) supplemented with 10% fetal bovine serum (Cellgro) and 1% penicillin/streptomycin (Gibco) in 150 mm dishes at 37° C. in a humidified 5% CO₂ incubator. In all cases, transfection efficiency was measured by transfection with pEGFP-N1 control vector (Clontech). Transient transfection (Chen et al., Mol. Cell Biol. 7:2745-2752, 1987) was performed in 24 well plates using cells plated 24 hrs prior to transfection at 3×10⁵ cells per well in 500 μl of media, corresponding to about 50 to 80% confluence, and used 1.5 μg total DNA and a 3:1 molar ratio of expression plasmid to respective reporter target plasmids. Cells were resuspended by trypsinization, and 5×10⁵ cells were washed with PBS, stained by incubation in 500 μl PBS containing 10 ng/μl propidium iodide (PI), and analyzed on an Influx flow cytometer (Cytopeia). Typically, 40,000 events were scored and gated first for log side and linear forward scatter to identify cells, and then for PI exclusion to identify viable cells for GFP fluorescence analysis. Transfections in the targeted gene correction assay were performed using Lipofectamine™ LTX (Invitrogen) according to the manufacturer's protocol. For each experiment, 225 ng of each of two plasmids was transfected; either donor and pBS-SK⁺ or donor and I-AniI expression plasmid. For the targeted gene correction assay, cells grown for approximately 72 hr after transfection were treated with trypsin and resuspended in PBS+2% formaldehyde. Cells were analyzed on an FACScan® flow cytometer (Becton Dickinson); 75,000 events were gated for linear side and forward scatter to identify cells; and GFP fluorescence was analyzed.

### Point Mutations to Generate an I-AniI Nickase.

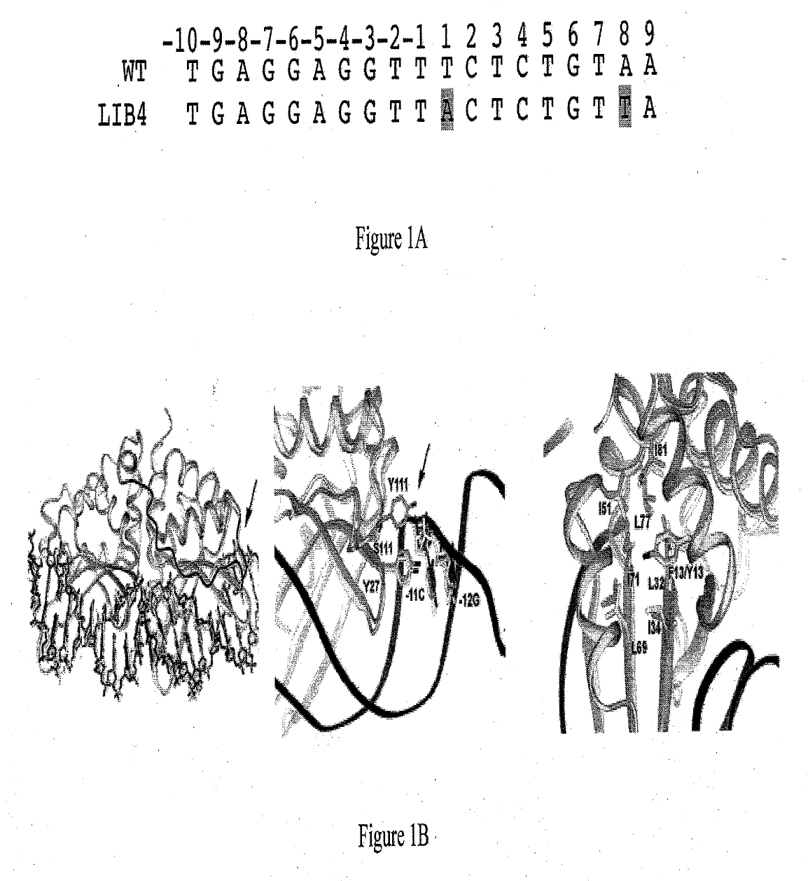

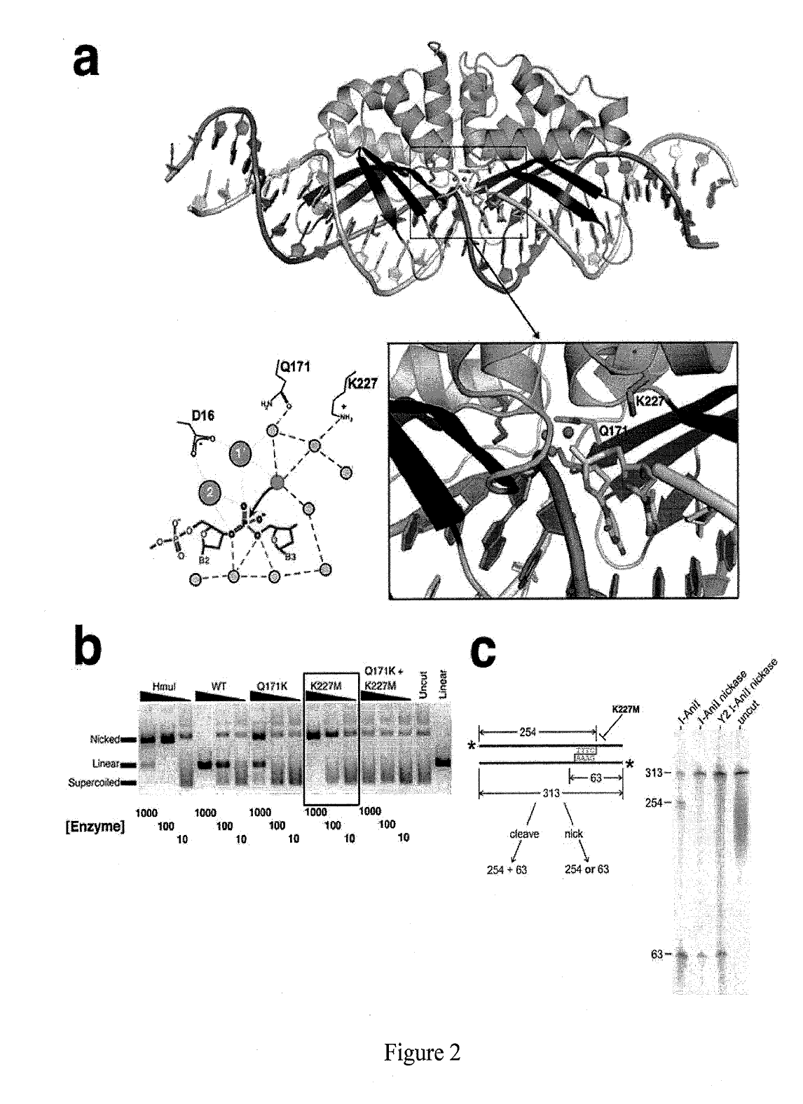

I-AniI, encoded by a group I intron harbored within the Aspergillus nidulans apocytochrome B oxidase gene, cleaves a 19 basepair asymmetric DNA target (FIG. 1A). To generate an I-AniI variant with strand-specific nicking activity, the enzyme's overlapping active sites were uncoupled by inactivating one catalytic center that is responsible for cleavage of a single DNA strand. At least three residues in I-AniI (the lysine residue at position 94 (K94) in the N-terminal domain, and the glutamine at position 171 and the lysine at position 227 (Q171/K227) in the C-terminal domain) are found in the periphery of its two active sites (FIG. 2A). Substitution of these amino acid residues with sterically similar side chains of differing chemical behaviors was intended to maintain active site structural integrity while locally disrupting the active site solvent network of one of the two I-AniI active sites. Activity was assayed using either a supercoiled plasmid substrate (pBlueScript) (FIG. 2B) or a synthetic, radio-labeled DNA duplex substrate (FIG. 2C), both containing the optimal DNA target sequence for I-AniI. Termed ‘LIB4’, this DNA sequence contains two basepair substitutions relative to the native target site of I-AniI that increase the enzyme's binding affinity and cleavage efficiency (Scalley et al., J. Mol. Biol. 372:1305-1319, 2007) (FIG. 1B). Preliminary analyses showed that mutation of K94 to methionine in the N-terminal active site (K94M) resulted in an enzyme construct that still exhibited significant double-strand break activity (not shown). In contrast, mutation of the C-terminal active site resulted in stronger nicking behavior. A construct corresponding to Q171K exhibited strong nicking activity, converting 60% of the plasmid substrate to nicked form in 2 hrs at 1 μM enzyme concentration, but retained significant residual double-strand cleavage activity. A second construct in this same active site, K227M, exhibited stronger nicking activity: at 1 μM enzyme, more than 99% of the plasmid substrate was nicked, with no detectable linearization after extended incubations. Finally, a double mutant harboring both the Q171K and K227M substitutions was completely inactive. Based on these results, the K227M variant was used for detailed studies.

In order to verify the stereochemistry and strand specificity of the K227M nickase reaction, digests were also performed using a radiolabeled, asymmetric linear DNA duplex containing the I-AniI target site (FIG. 2C). I-AniI K227M nickase clearly generated single strand products of the predicted sizes with little or no background cleavage of the opposing DNA strand.

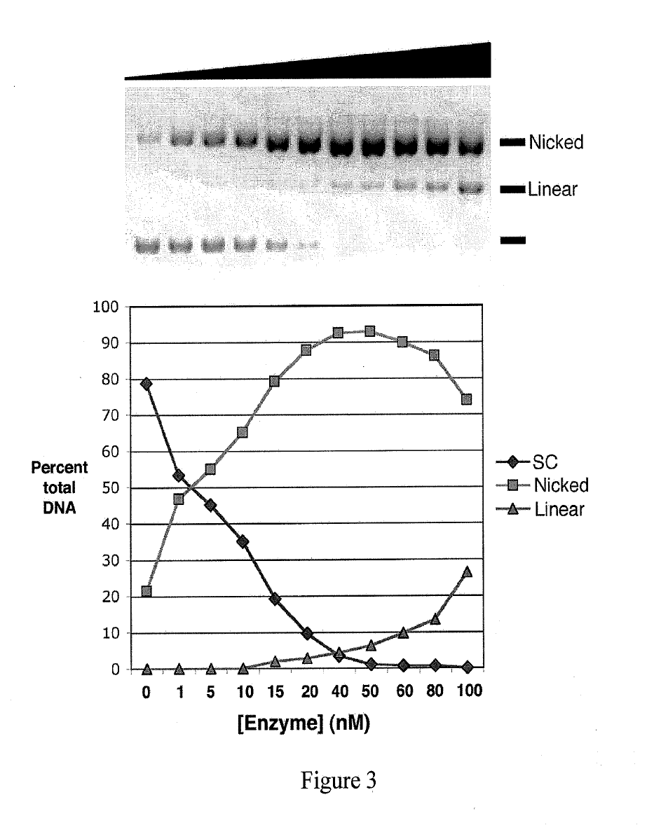

The K227M mutation was also incorporated into a variant of the I-AniI protein scaffold that harbors two additional mutations (a tyrosine replacement of phenylalanine at position 13 (F13Y) and a tyrosine substitution of serine at position 111 (S111Y), both located far from the active sites; (FIG. 1B) that increase DNA binding affinity and cleavage activity at physiological temperatures. When placed into this ‘Y2’ enzyme scaffold, the K227M construct displayed site-specific nicking activity at significantly lower enzyme concentrations (1 to 10 nM; FIG. 3). At higher enzyme concentrations, a measurable (although still minor) fraction of the substrate was eventually converted to linearized product.

### Comparison of In Vitro Activities of Native I-AniI and Engineered Nickase.

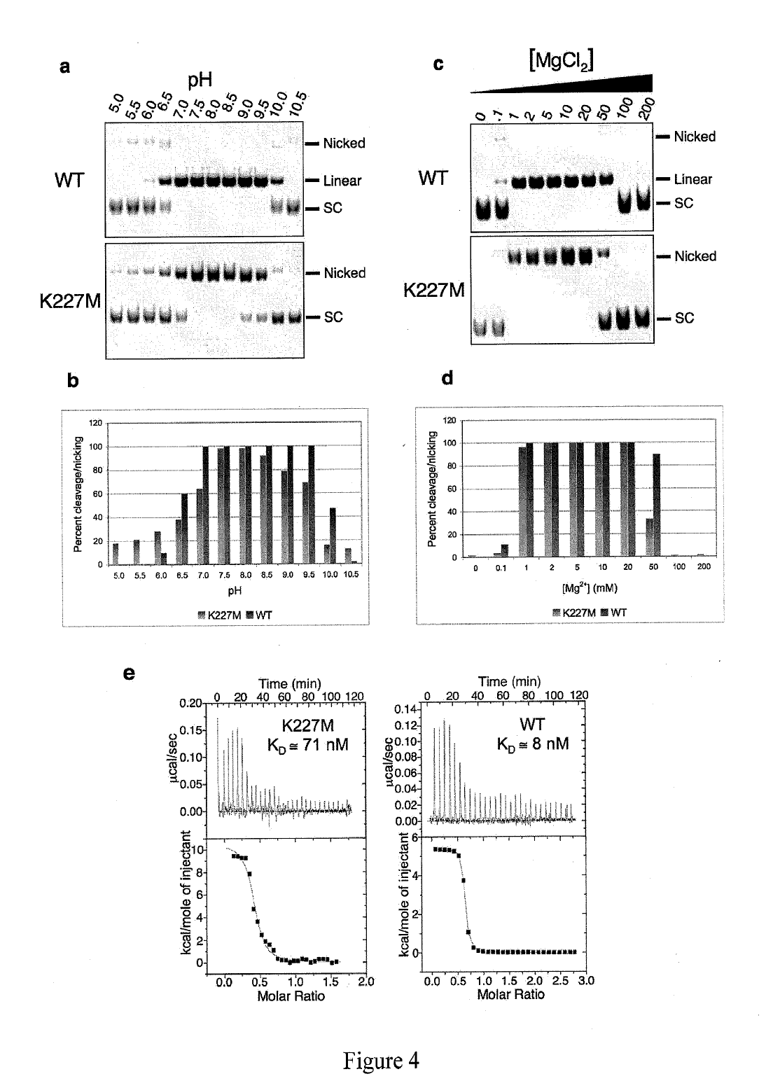

Relative DNA cleavage activity by wild-type I-AniI and the K227M mutant was measured over a pH range of 5.0 to 10.5. At protein concentrations in excess of the enzymatic KD, wild-type I-AniI exhibited optimal activity at pH 8.5 to 9.5; the I-AniI K227M nickase exhibited a similar, but slightly shifted pH-profile, with maximal activity in a range of pH 7.5 to 8.5 (FIGS. 4A and 4B). Comparison of activities of these two enzymes at 0 to 50 mM MgCl₂ showed that both wild-type I-AniI and I-AniI K227M exhibited optimal activity in a range from 1 to 20 mM MgCl₂ (FIGS. 4C and 4D).

Isothermal titration calorimetry (ITC) was used to determine the dissociation constants of native and nicking I-AniI binding to the optimal LIB4 DNA target site (FIG. 4E). Calorimetric experiments were conducted as described previously (Eastberg et al., Biochemistry 46:7215-25, 2007). Average values were obtained for the enzyme dissociation constant (K_{D}) of 8 nM for the wild-type enzyme, 71 nM for the nicking variant (K227M), and 62 nM for the catalytically inactive variant (K227M/Q171K). The nickase exhibits an increase of +1.4 kcal/mol in ΔG_{binding} compared to the wild type protein. This difference is primarily due to a +5.8 kcal/mol increase in enthalpy change upon nickase binding, implying that the nicking variant has lost contacts to the DNA target—presumably because a hydrogen bond formed by the K227 side chain to the DNA phosphate backbone (Scalley-Kim et al., J. Mol. Biol. 372:1305-1319, 2007) is eliminated.

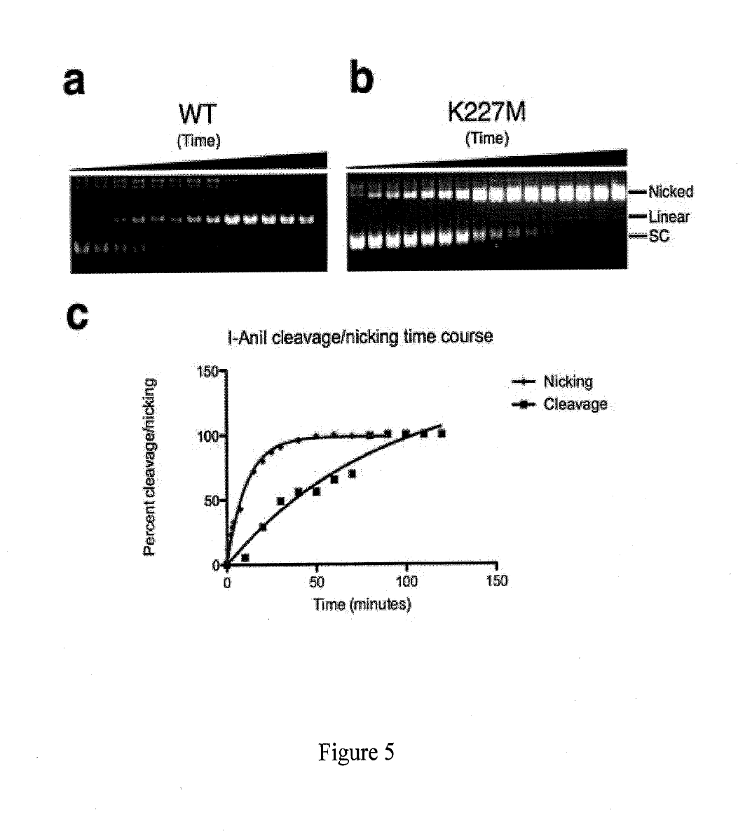

The relative rates of DNA cleavage by the native and nicking I-AniI were then determined (FIG. 5A through 5C). The experiments were performed at 1 μM enzyme concentrations, exceeding the native and nickase dissociation constants by approximately 100- and 10-fold respectively, and thereby reporting on differences in the rate-limiting steps of the reactions. The rate of the nicking reaction was approximately 8-times faster than the wild-type cleavage reaction, exhibiting a t_{1/2} of 7.6 min versus 59.6 min for the native enzyme.

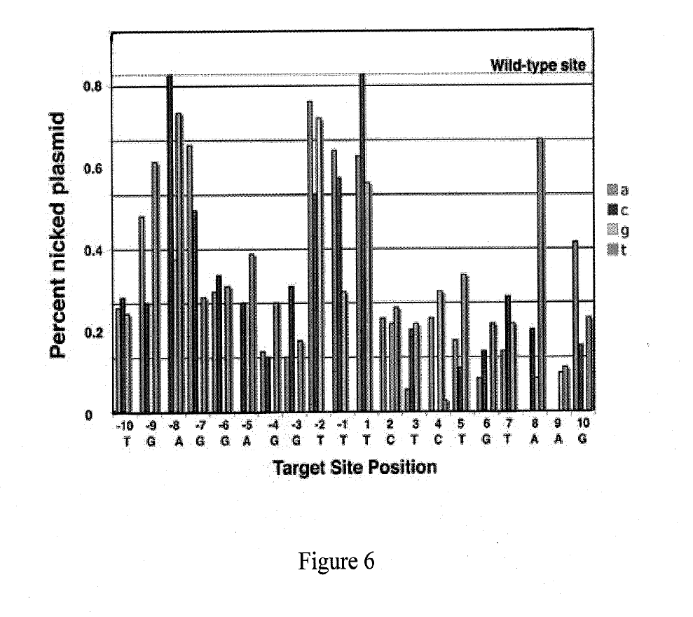

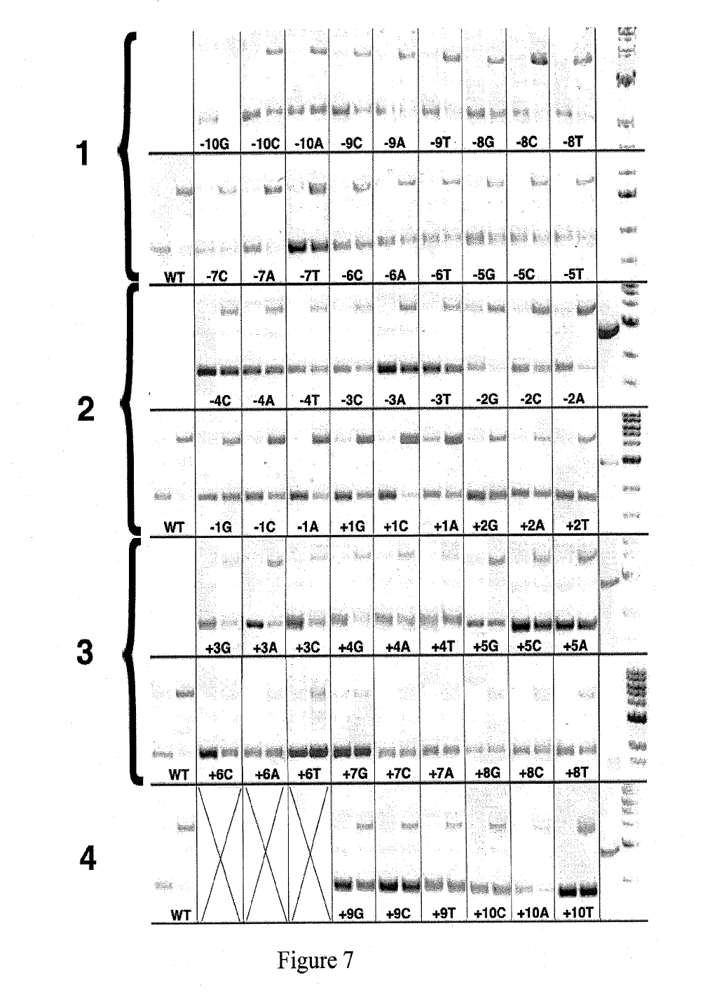

The overall specificity of the native enzyme and the nickase was then compared (FIG. 6). A previous analysis of I-AniI site specificity, using a randomized site library, estimated the overall specificity of I-AniI to be approximately 1 in 10⁸ under ideal cleavage conditions (Scalley et al., J. Mol. Biol. 372: 1305-1319, 2007). A similar screen of a randomized site library was not possible for the engineered nicking enzyme variants described in this study, due to the low background of nonenzymatically nicked substrates in plasmid preparations. Therefore, the specificity profile was measured by assaying the relative nicking of every possible single base pair variant of the I-AniI target site. This corresponded to 60 mutated sequences, comprising all three possible basepair substitutions at each of 20 positions across the target (FIG. 7). The specificity profile of the I-AniI K227M nickase is quite similar to that observed previously for the wild-type enzyme. Specificity was greatest across basepairs +/−3, 4, 5 and 6 in each half-site, where the enzyme makes the majority of its base-specific contacts in the DNA major groove. The enzyme was least specific across three out of four bases at the target's center, positions −2 to +1, where the enzyme straddles the DNA minor groove and predominantly contacts the DNA backbone. It also exhibited low specificity at the outer flanks of each half-site where the enzyme makes less saturated contacts to individual bases. The similarity between wild-type and nicking I-AniI specificity profiles indicated that the K227M substitution has not dramatically affected target site recognition by the enzyme.

### Comparison of Recombination In Vivo Stimulated by the I-AniI Cleavage or Nicking Activities.

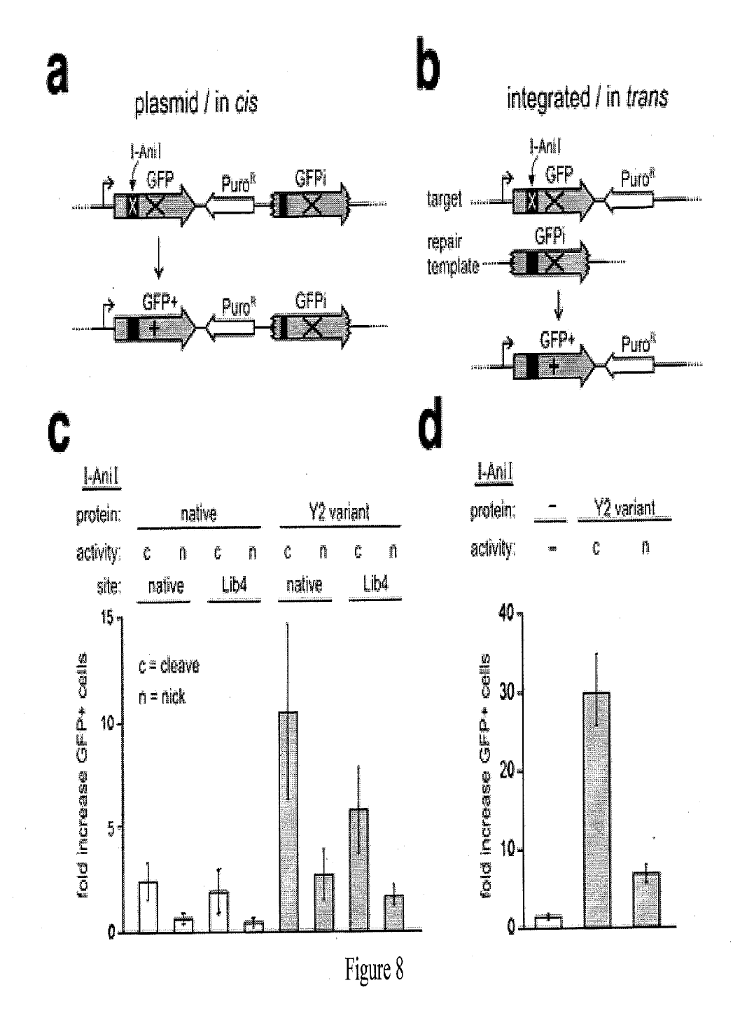

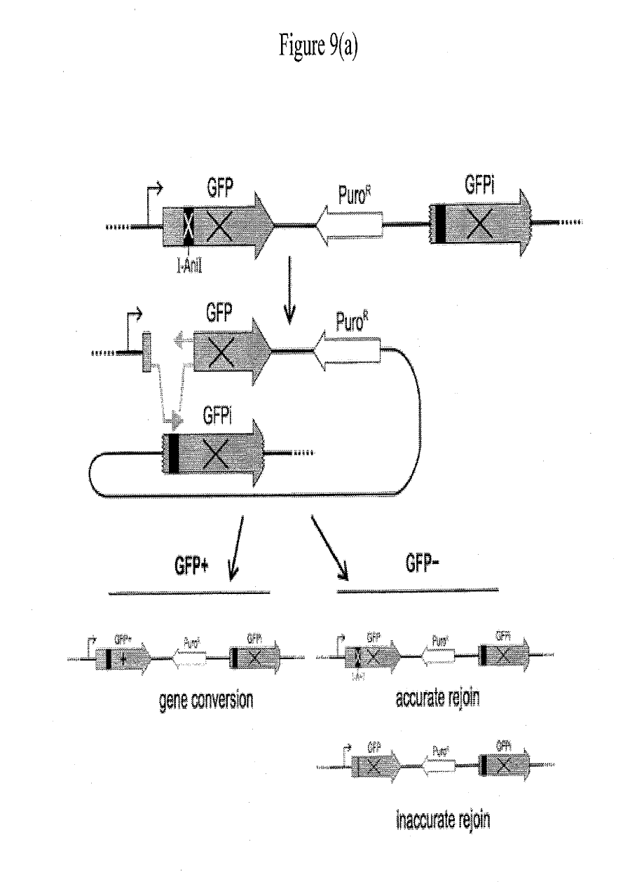

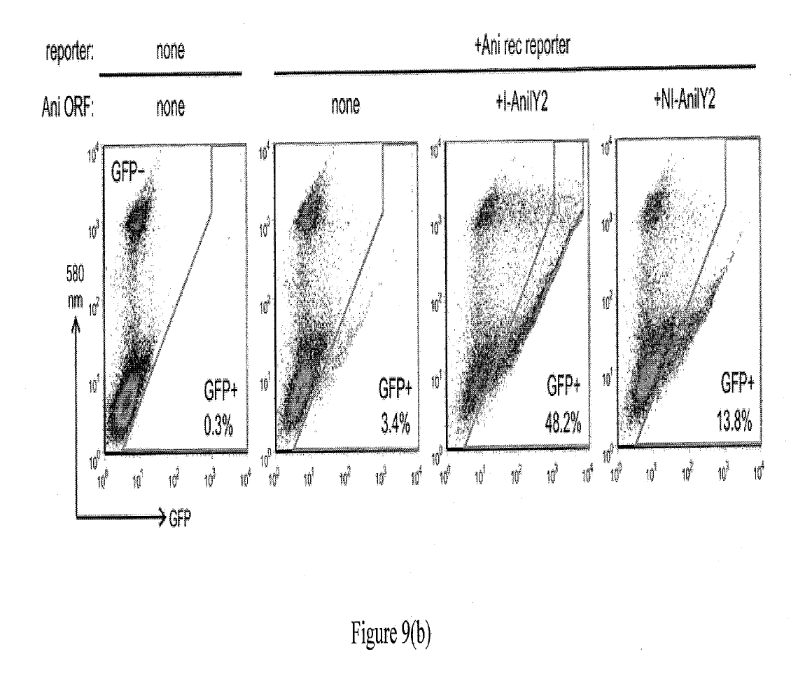

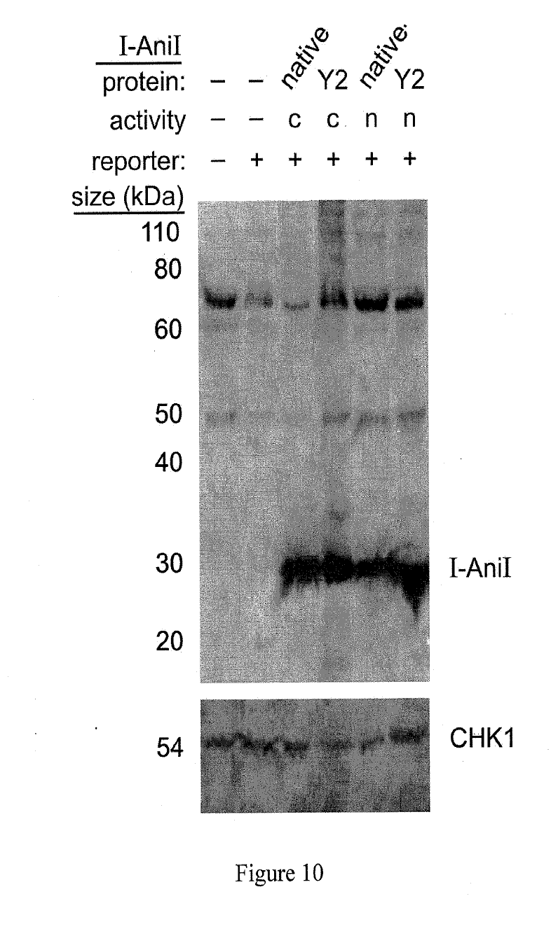

Reporter assays were developed to measure either intramolecular recombination in cis, or recombination of a chromosomal target mediated by a donor in trans. The assay for recombination in cis used a reporter, DR-GFPAni, based on the direct repeat recombination reporter plasmid (DR)-GFP (Pierce et al., Genes Dev. 13:2633-2638, 1999), which was designed to score only gene conversion, and not repair mediated by NHEJ or single-strand annealing. This plasmid reporter, which does not contain elements known to drive replication in human cells, carries two disabled GFP genes, driven by an upstream cytomegalovirus (CMV) promoter (FIG. 8A; FIG. 9A and Table 1). Human 293T fibroblasts were transiently cotransfected with DR-GFPAni reporter plasmids containing an I-AniI WT or Lib4 variant target site, together with vectors expressing the I-AniI cleavase, nickase, or inactive enzyme in the wild-type (WT) or I-AniI Y2 protein scaffold. Transfection efficiency was consistently greater than 90%. GFP⁺ cells were identified and quantified by fluorescence-activated cell sorting (FACS), and western blotting confirmed comparable levels of expression of I-AniI variants (FIG. 10). The fraction of GFP⁺ cells ranged from a background of about 3 to about 5% for cells transfected with the reporter alone, up to about 50% in cells transfected simultaneously with the reporter plasmid and an I-AniI Y2 cleavase expression plasmid (FIG. 8C and FIG. 10). Expression of I-AniI Y2 cleavase resulted in a 10-fold stimulation of recombination at the native site, and 6-fold stimulation at the Lib4 site. Expression of the I-AniI nickase stimulated recombination 2.5-fold (FIG. 8C), or approximately one-fourth as efficiently as the I-AniI cleavase. The DR-GFPAni reporter plasmid does not contain elements known to drive replication in human cells, so it is unlikely that recombination in this assay is initiated at DSBs generated during DNA replication, rather than by nicks.

TABLE 1
Frequency of GFP⁺ recombinant cells in plasmid/integrated targeting experiments.
1. Plasmid - in cis targeted recombination experiments:

| | | | | |
|---|---|---|---|---|
| | | # exper- | Mean | ±SD |
| Reporter/Target site | Endonuclease² | iments | GFP⁺ (%) | (%) |

Series 1¹

| | | | | |
|---|---|---|---|---|
| none (cells alone) | None | 3 | 0.20 | 0.10 |
| EGFP-C1 (positive control) | None | 3 | 91.83 | 2.19 |
| pDRGFP/native | None | 3 | 5.10 | 2.21 |
| pDRGFP/Lib4 | None | 3 | 5.93 | 1.70 |
| pDRGFP/native | I-AniI | 3 | 11.03 | 0.58 |
| pDRGFP/Lib4 | I-AniI | 3 | 10.37 | 3.23 |
| pDRGFP/native | Y2 I-AniI | 3 | 47.47 | 2.29 |
| pDRGFP/Lib4 | Y2 I-AniI | 3 | 32.23 | 1.54 |
| pDRGFP/native | I-AniI nickase | 3 | 2.93 | 0.15 |
| pDRGFP/Lib4 | I-AniI nickase | 3 | 2.97 | 1.96 |
| pDRGFP/native | Y2 I-AniI nickase | 3 | 12.40 | 1.85 |
| pDRGFP/Lib4 | Y2 I-AniI nickase | 3 | 9.40 | 1.51 |

Series 2¹

| | | | | |
|---|---|---|---|---|
| none (cells alone) | None | 3 | 0.77 | 0.51 |
| EGFP-C1 (positive control) | None | 3 | 98.83 | .21 |
| pDRGFP/native | None | 3 | 11.5 | 2.54 |
| pDRGFP/native | I-AniI | 3 | 20.00 | 3.73 |
| pDRGFP/native | Y2 I-AniI | 3 | 58.40 | 3.99 |
| pDRGFP/native | I-AniI nickase | 3 | 3.83 | 1.53 |
| pDRGFP/native | Y2 I-AniI nickase | 3 | 20.13 | 1.89 |
| pDRGFP/native site | Y2 I-AniI nickase | 3 | 15.10 | 2.51 |
| | dead mutant | | | |

2. Integration - in trans targeted recombination experiments:

| | | | | | |
|---|---|---|---|---|---|
| | | | # exper- | mean | ±SD |
| Reporter³ | Donor⁴ | Endonuclease² | iments | GFP⁺ (%) | (%) |

Series 1

| | | | | | |
|---|---|---|---|---|---|
| pZF-GFPAni | GFPt | none | 3 | 0.0020 | 0.00081 |
| pZF-GFPAni | none | Y2 I-AniI | 4 | 0.0019 | 0.00072 |
| pZF-GFPAni | none | Y2 I-AniI nickase | 4 | 0.0022 | 0.0015 |
| pZF-GFP-Ani | GFPt | Y2 I-AniI | 4 | 0.059 | 0.024 |
| pZF-GFPAni | GFPt | Y2 I-AniI nickase | 4 | 0.016 | 0.0032 |

Series 2:

| | | | | | |
|---|---|---|---|---|---|
| pZF-GFPAni | GFPt | none | 2 | 0.0014 | 0.000041 |
| pZF-GFPAni | none | Y2 I-AniI | 2 | 0.0016 | 0.00033 |
| pZF-GFPAni | none | Y2 I-AniI nickase | 2 | 0.0016 | 0.00022 |
| pZF-GFP-Ani | GFPt | Y2 I-AniI | 2 | 0.048 | 0.018 |
| pZF-GFPAni | GFPt | Y2 I-AniI nickase | 2 | 0.012 | 0.0037 |
| pZF-GFPAni | none | Y2 I-AniI nickase | 2 | 0.0016 | 0.00031 |
| | | dead mutant | | | |
| pZF-GFPAni | GFPt | Y2 I-AniI nickase | 2 | 0.0021 | 0.00094 |
| | | dead mutant | | | |

¹The two different series were performed as described in this example. The cis Series 1 assays were performed with propidium iodide (PI) counterstaining prior to flow cytometry, whereas Series 2 did not include this additional staining step. Series 2 data for both types of assay are plotted in FIG. 4.
²The I-AniI endonuclease proteins used were: I-AniI HyperK (here listed as I-AniI) containing a silent G25G silent mutation to disrupt a cryptic splice site together with F80K and L232K substitutions, the Y2 variant of I-AniI that includes F13Y and S111Y substitutions; and a catalytically inactive “dead” mutant of Y2 I-AniI nicase that includes an additional active site Q171K substitution in addition to the nickase K227M substitution.
^{3,4}The integrated reporter plasmid pZF-GFPAni consists of the 5′ end of the pDR-GFPAni containing an I-AniI Lib4 target site and an adjacent 4 kb poly-lacO array inserted downstream of the GFP cassette. A clonaly derived 293T subline containing the chromosomally integrated reporter was used as a repair target, and a truncated 3′ GFP cassette from pDR-GFPAni as a transfected repair template (GFPt) in trans recombination experiments.

Single-Strand Nicks Induce Homologous Recombination with Less Toxicity than Double-Strand Breaks Using an AAV Template

As discussed above, the enhancement of homologous recombination (HR) using targeted double-strand breaks (DSBs) has the potential to correct gene defects in their endogenous loci, avoiding many problems which have plagued traditional gene therapy. However, a DSB by definition is a DNA damaging event, and resulting toxicity and potential for mutagenesis and translocations are serious problems for this strategy. In the present example the ability of nicks and DSBs to enhance homologous recombination were compared using the homing endonuclease I-AniI and the redesigned variant which produces only single-strand nicks.

For tissue culture, Human Embryonic Kidney 293 and 293T cells were grown using Dulbecco's modified Eagle medium (DMEM) plus 10% Hyclone Cosmic Calf Serum (Thermo Scientific) at 37° C. with 10% CO₂.

Plasmid Construction: Target plasmids pCnZPNOA2 and pCnZPNOA3 were generated by site-directed mutagenesis of pCnZPNO using primers AnidE2F (cgctgatcctttgcTTACAGAGAAACCTCCTCAtacgcccacgcgatg) (SEQ ID NO: 15) and AnidE2R (catcgcgtgggcgtaTGAGGAGGTTTCTCTGTAAgcaaaggatcagcg) (SEQ ID NO: 16) for pCnZPNOA2, and AnidE3F (gctgatccttTTACAGAGAAACCTCCTCAtgggtaacagtcttg) (SEQ ID NO: 17) and AnidE3R (caagactgttacccaTGAGGAGGTTTCTCTGTAAaaggatcagc) (SEQ ID NO: 18) for pCnZPNOA3. Plasmid pExodusY2 expressing an HA-tagged DSB-inducing I-AniY2 is a plasmid based on the pcDNA3.1 backbone with the HA-tagged I-AniIY2 construct with a second generation nuclear localization signal. The I-AniIY2 enzyme is expressed from the CMV promoter in the pcDNA3.1 backbone. The function of this plasmid is to express the I-AniIY2 enzyme in transfected cells and a plasmid that expresses the correct I-AniIY2 enzyme will suffice. Lentivirus construct pRRLsinExY2imC (pRRLSIN.SFFV.HA.2ndGenNLS.reoAniY2.1RES.mCherry) expressing an HA-tagged DSB-inducing I-AniIY2 and mCherry is a self-inactivating lentiviral vector (RRLSIN), with an SFFV promoter driving expression of an HA tagged I-AniIY2 with a second generation nuclear localization signal (NLS), followed by the fluorescent marker mCherry driven from the SFFV promoter through an internal ribosome entry site (ires). The open reading frame of the I-AniIY2 gene in both pExodusY2 and pRRLsinExY2imC are identical. The purpose of this plasmid is to generate a lentivirus that expresses the HA-tagged I-AniIY2 enzyme with a nuclear localization signal with a marker to titrate the virus, and a similar retrovirus vector which satisfies these conditions will suffice.

The K227M nicking variants (pnExodusY2 and pRRLsinnExY2imC) were generated using site-directed mutagenesis with primers nExodusK227M-F (gttaggcaacATGaaactgcaatac) (SEQ ID NO: 19) and nExodusK227M-R (gtattgcagtttCATgttgcctaac) (SEQ ID NO: 20), and the K227M/E148Q catalytically inactive variants (pdExodusY2 and pRRLsindExY2imC) were generated using the above primers as well as dExodusE148Q-F (gatttatagaagctCAGggctgtttcag) (SEQ ID NO: 21) and dExodusE148Q-R (ctgaaacagccCTGagcttctataaatc) (SEQ ID NO: 22). Empty lentivirus vector pRRLsinXimC was generated by removing an RsrII/SbfI fragment containing the I-AniIY2 gene from pRRLsinExY2imC.

Generation of target cells: The foamy virus vectors used to insert the inactive lacZ target containing an I-AniI recognition site was generated by transfection of 293T cells with foamy virus production plasmids pCINGSΔΨ, pCINPS, pCINES and either pCnZPNOA2 or pCnZPNOA3. These plasmids function to express foamy virus proteins (pCINGSΔΨ, gag; pCINPS, pol; and pCINES, env). These plasmids are based on the published plasmids for foamy vector (Trobridge, et al., Methods Enzymol 346: 628-48 (2002)). The construction and use of such third generation foamy virus plasmids have been reviewed in Trobridge G D. Expert Opin Biol Ther. 2009 November; 9 (11):1427-36. Any functional third generation foamy vector helper plasmids could be used to generate the foamy vectors used. Supernatant was collected and filtered (0.45 μm). Polyclonal populations of target cells were generated by plating 2.5×10⁵ 293 cells in a 6 cm dish and transducing the cells with one of the foamy virus vectors the next day. Two days post-transduction cells were split into media containing 400 μg/ml G418 for selection of transduced cells. Each polyclonal population consisted of ˜50-200 individual integration events.

Transfection HR assay: Target cells (293/CnZPNOA2 and 293/CnZPNOA3) were plated at 2×10⁶ cells per 6 cm dish on day 0 and transfected on day 1 with 5 μg of the lacZ repair template plasmid (pA2nZ3113) and 5 μg of one of the I-AniI expression plasmids (pExodusY2, pnExodusY2, pdExodusY2, or pLXL-gfp as a control) per 6 cm dish. Media was changed on day 2 and plates were fixed and stained for β-gal expression on day 3.

Generation of lentivirus vectors: 293T cells were transfected with lentivirus production plasmids (pMDG and pCMVΔR8.2) and one of the I-AniI expression vectors (pRRLsinExY2imC, pRRLsinnExY2 imC, pRRLsindExY2 imC, or empty vector pRRLsinXExY2imC). Supernatant was collected and filtered (0.45 μm). To quantify virus titers, 293 cells were plated at 5×10⁴ cells per well of a 24 well plate. Cells were transduced with 20 μl of each virus the next day and titer was determined by quantification of mCherry-expressing cells using flow cytometry two days after transduction.

Production of AAV template for HR: AAV vectors were produced by plating 4×10⁶ 293 cells per 10 cm dish and transfecting the next day with the AAV2 production plasmid pDG and AAV vector plasmid pA2-nZ3113 (20 μg and 10 μg per dish respectively). Cells and supernatant were collected and purified using a heparin column (Halbert, Methods Mol Biol 246:201-12 (2004)). Quantification was determined by Southern Blot of DNA extracted from the purified AAV prep.

AAV HR assay and titration of I-AniI toxicity: On day 0, 5×10⁴ target cells per well were plated in 24 well dishes, and transduced with one of the I-AniI-expressing lentivirus vectors at varying MOIs on day 1. On day 3, 4×10⁵ lenti-transduced cells were plated into a new 24 well dish, and infected with AAV2-nZ3113 on day 4 at an MOI of 1.5-2×10⁴ vector genomes per cell. On day 5, 0.25% of the cells were plated in a 10 cm dish in order to count the number of viable cells, and 99.75% of cells were plated in a 15 cm dish to measure HR. Media was changed on day 9. On day 13 the 0.25% plate was fixed and surviving colonies were counted after Coomassie staining, and the 95% plate was fixed and stained for β-gal expression.

The results showed that when both template and I-AniI expression plasmids were transfected into 293 cells containing an integrated copy of an inactive lacZ target, the nickase enhance homologous recombination up to 300-fold above transfection of template plasmid alone (compared to 8,000-fold enhancement with the double-strand breaks-inducing enzyme). When the template was delivered with an AAV vector and the endonuclease construct with a lentivirus for longer expression, it was found that both double-strand breaks and nicks enhance homologous recombination in a manner dependent on the amount of endonuclease used. While homologous recombination was induced with lower amounts of double-strand break-inducing enzyme than with nick-inducing enzyme, the toxicity observed with the double-strand break-inducing enzyme was far more severe (>80% cell death at an MOI of 1). In contrast, the toxicity of the nicking endonuclease was low and was not distinguishable from the toxicity of an inactive endonuclease or the toxicity of an empty lentivirus vector expressing only the mCherry marker used to titer vectors. Due to the double-strand break toxicity, the maximum amount of homologous recombination observed with nicks and with double-strand breaks was similar (20-60 nick-induced foci compared to 50-80 double-strand breaks-induced foci in 5×10⁴ cells).

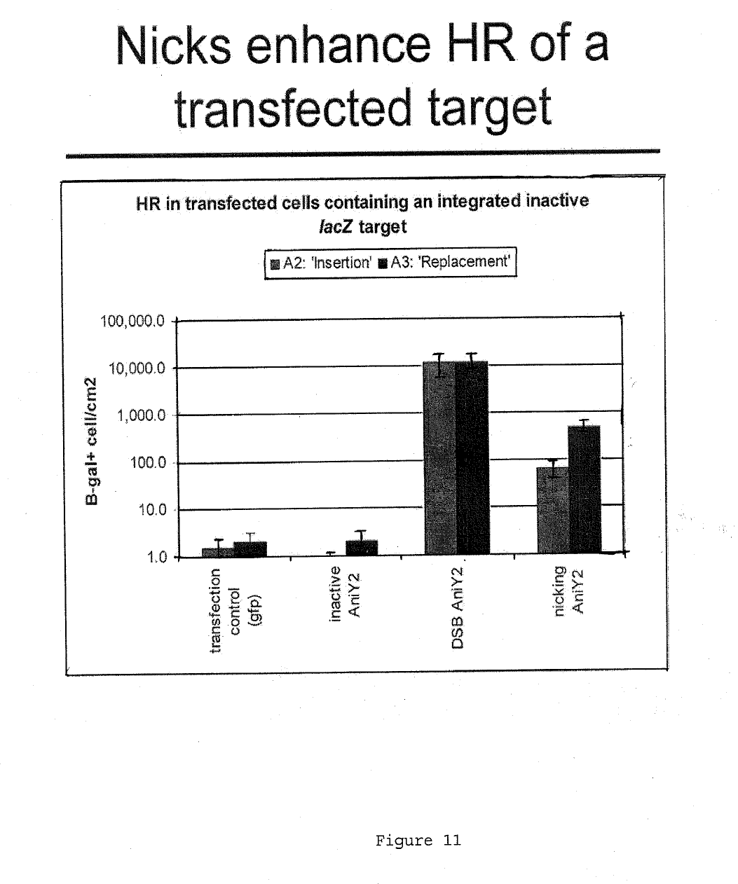

For both assays, two different lacZ target sites were investigated: one in which the 19 bp I-AniI recognition site replaced a 19 bp region in the lacZ gene, and the other in which the I-AniI site was inserted into the same location. The lacZ target with the “replacement” inactivating mutation supported nick-induced homologous recombination at a 10-fold higher rate than the target with the “insertion” mutation in both the AAV assay and the transfection assay, while no difference in double-strand breaks-induced homologous recombination was observed between the two targets. The results are displayed in FIG. 11.

Both the observation that nickases can stimulate homologous recombination with lower toxicity than double-strand breaks and the observation that target site design affects nick-induced homolgous recombination but not double-strand break-induced homologous recombination strongly argue that nicks induce homologous recombination through a different mechanism than double strand breaks.

Therefore, the present invention for inducing homologous recombination with a nicking enzyme (nickase) and a viral delivery system finds use in clinical gene therapy applications, allowing for more efficient gene correction without the toxicity and mutagenic activity of double-strand breaks.

A reporter assay was also devised for gene correction in trans. In this assay, an exogenous truncated GFP donor gene was used to correct an inactive chromosomal GFP transgene (FIG. 8B). 293T cells carrying chromosomally integrated copies of this defective GFP gene were generated by transfection of pZF-GFPAni plasmid DNA followed by selection for puromycin-resistance to recover clonal integrants. To assay repair mediated by a donor in trans, cells carrying this chromosomally integrated defective GFP gene were cotransfected with the truncated GFP gene donor and a vector expressing I-AniI Y2 cleavase, nickase, or inactive enzyme. Transfection of the donor in the absence of Ani expression resulted in generation of essentially no GFP⁺ cells (about 1 to 3×10⁻⁵). Expression of the I-AniI Y2 cleavase increased gene correction 30-fold, and expression of the I-AniI Y2 nickase increased gene correction 8-fold (FIG. 8D and Table 1). The four-fold difference between stimulation of recombination in trans by the I-AniI Y2 cleavase and nickase parallels the difference observed in assays of recombination in cis, despite higher background levels observed in cis.

The I-AniI endonuclease described in the present invention was converted to a nickase, without a significant reduction in the enzyme's specific activity or site-specificity. I-AniI nickase was very active, nicking its DNA target site approximately 8-fold faster than wild-type I-AniI generated a double-strand break (DSB). Thus, the nickase variant maintains—or improves upon—the activity and specificity of native I-AniI, to provide a protein with properties desirable for therapeutic application.

Conversion of native I-AniI to a corresponding nickase takes advantage of a natural asymmetry in DNA cleavage that is often displayed by monomeric LAGLIDADG homing endonucleases. The algal endonuclease I-CpaII displays metal ion-dependent asymmetric cleavage, preferentially nicking the bottom strand of its target site at very low magnesium concentrations (Turmel et al., Nucl. Acids Res. 23:2519-2525, 1995). The yeast homing endonuclease I-SceI has higher affinity for binding to the 3′ DNA half-site, leading to accumulation of nicked intermediates during the cleavage reaction (Perin et al., EMBO Journal, 12:2939-2947. 1993). Finally, the archaeal endonuclease I-DmoI preferentially cleaves the coding strand of its host gene (Aggard et al., Nucl. Acids Res. 25:1523-1530, 1997), a preference which can be further enhanced by mutation of the LAGLIDADG (SEQ ID NO: 1) motif (Silva et al., Nucl. Acids Res. 32:3156-3168, 2004).

The mutational strategy disclosed herein was governed by the catalytic mechanism of these endonucleases. Whereas mutation of metal-binding residues within the LAGLIDADG (SEQ ID NO: 1) motif causes significant disruption of the endonuclease active site and loss of DNA binding affinity, mutation of the more peripheral polar side chains involved in solvent-mediated interactions and proton transfer (Q47 and K98 in I-CreI; Q171 and K227 in the C-terminal domain of I-AniI) causes significant reductions in catalytic efficiency with little effect on either overall affinity or the structure of the enzyme-DNA complex (Chevalier et al., Biochemistry 43:4015-4026, 2004). In the enzyme I-SceI, substitution of pseudo-symmetric residues K122 and K223 functioned as a nickase (Niu et al., J. Mol. Biol. 382:188-202, 2008), but those mutant enzymes generated significant fractions of linearized product under extended digest conditions in contrast with the K227M I-AniI variant. Given the loosely conserved nature of peripheral side chains in LAGLIDADG endonuclease active sites and the use of a solvent network for deprotonation of the nucleophile rather than a single explicit protein side chain, it is clear that different LAGLIDADG endonuclease active sites display variable amounts of mechanistic redundancy in their ability to carry out acid/base catalysis, with I-SceI retaining function even in the absence of the primary general base in either active site.

I-AniI nickase was able to stimulate homologous recombination in human cells at an efficiency approximately one-fourth that of the wild-type enzyme, both when measuring transient recombination in cis, and in assays of recombination of a chromosomal target gene in trans. Recombination initiated by nicks as opposed to double-strand breaks has not been well studied, in large part due to a paucity of reagents that reliably nick DNA in vivo in a site-specific fashion. The availability of variants of LAGLIDADG enzymes I-SceI (Niu et al., J. Mol. Biol. 382:188-202, 2008) and I-AniI, that can initiate recombination by nicking or cleaving the respective target site, should facilitate mechanistic analyses of nick or break processing that leads to the generation of recombinant molecules.

Inter-molecular recombination in trans initiated by homing endonuclease target site cleavage or nicking provides a useful way to promote homology-dependent targeted gene correction in vivo. A sequence-specific nickase, such as the I-AniI variant described herein, has particular use for therapeutic applications, including the targeted repair of human disease-causing mutations. Although DSBs may stimulate homologous recombination more efficiently than nicks, they are also more likely to promote mutagenic repair or potentially deleterious genome rearrangements at the endonuclease-induced break site (Niu et al., J. Mol. Biol. 382:188-202, 2008; Rouet et al., Proc. Natl. Acad. Sci. 91:6064-6068, 1994; Monnat et al., Biochem. Biophys. Res. Commun. 255:88-93, 1999; Guirouilh-Barbat et al., Mol. Cell, 14:611-623, 2004; Allen et al., DNA Repair (Amst), 2:1147-1156, 2003). Many of these deleterious events may be avoided by using site-specific nicking as described herein, as opposed to cleavage to target and initiate recombinational repair in living cells. Thus engineered, site-specific nickase variants of I-AniI and other homing endonucleases are particularly useful in targeted genome engineering as well as therapeutic, targeted gene repair

1. A variant single amino acid chain homing endonuclease comprising a single functional active site which can cleave a single strand of a double-stranded polynucleotide and which maintains the DNA substrate specificity of the wild-type single chain homing endonuclease.

2. The variant homing endonuclease according to claim 1, wherein the basic amino acid for proton transfer and solvent activation in one active site is replaced with a non-functional amino acid residue.

3. The variant homing endonuclease according to claim 1, wherein the variant homing endonuclease is derived from a LAGLIDADG homing endonuclease.

4. The variant homing endonuclease according to claim 3, wherein the LAGLIDADG homing endonuclease is I-AniI, I-SceI, I-ChuI, I-CreI, I-CsmI, PI-TliI, PI-MtuI, I-CeuI, I-SceII, I-SceIII, HO, PI-CivI, PI-CtrI, PI-AaeI, PI-BsuI, PI-DhaI, PI-DraI, PI-MavI, PI-MchI, PI-MfuI, PI-MflI, PI-MgaI, PI-MgoI, PI-MniI, PI-MkaI, PI-MleI, PI-MmaI, PI-MshI, PI-MsmI, PI-MthI, PI-MtuI, PI-MxeI, PI-NpuI, PI-PfuI, PI-RmaI, PI-SpbI, PI-SspI, PI-FacI, PI-MjaI, PI-PhoI, PI-TagI, PI-ThyI, PI-TkI, PI-TspI, and I-MsoI.

5. The variant homing endonuclease according to claim 4, wherein the LAGLIDADG homing endonuclease is I-AniI.

6. The variant I-AniI according to claim 5, wherein the lysine at position 227 is replaced with a non-functional amino acid.

7. The variant I-AniI according to claim 6, wherein the non-functional amino acid is methionine, alanine, glutamine, asparagine, or leucine.

8. The variant I-AniI according to claim 7, wherein the non-functional amino acid is methionine.

9. The variant homing endonuclease according to claim 1, wherein the variant homing enzyme further comprises modification of amino acid residues to change the site specificity from the wild-type sequence, or that has been modified to improve target site binding efficiency or some other characteristic of the enzyme.

10. The variant homing endonuclease according to claim 9, wherein the variant homing endonuclease is I-AniI and the amino acid modification is a mutation of the amino acid residue at positions 13 and 111 to tyrosine.

11. The variant homing endonuclease according to claim 10, wherein the phenylalanine at position 13 and the serine at position 111 are changed to tyrosine and the binding affinity and cleavage activity are increased as compared with a wild-type amino acid sequence of I-AniI.

12. The variant homing endonuclease according to claim 9, wherein the mutation in the enzyme improves the solution behavior of the enzyme.

13. A nucleic acid sequence that encodes a variant homing endonuclease of claim 1.

14. The nucleic acid sequence according to claim 13, wherein the sequence further encodes a promoter, a transcriptional activator region, and a translational regulator region.

15. A vector comprising the nucleic acid sequence that encodes a variant homing endonuclease of claim 13.

16. The vector according to claim 15, wherein the nucleic acid encodes a detectable label.

17. The vector according to claim 16, wherein the detectable label is green fluorescent protein.

18. The vector according to claim 17, wherein the protein encoded is a fusion protein.

19. A method for stimulating homologous recombination at a target site in a cell comprising contacting the cell with a vector according to claim 15 under conditions by which the vector expresses the variant homing endonuclease and stimulates homologous recombination at the target site recognized by the homing endonuclease.

20. The method according to claim 19 for stimulating homologous recombination at a target site in a cell, wherein the vector is a viral vector.

21. The method according to claim 20 for stimulating homologous recombination at a target site in a cell, wherein the viral vector is selected from the group consisting of a retrovirus, lentivirus, adenovirus, adeno-associated virus, vaccinia virus, and herpes simplex virus.

22. A method for targeting and initiating homologous recombinational repair to a nucleotide sequence in a cell, comprising contacting the cell with a vector according to claim 15 and a homologous sequence non-identical to the cell sequence, under conditions by which the vector expresses the variant homing endonuclease and stimulates recombinational repair with the homologous non-identical sequence at the target site that is recognized by the homing endonuclease.

23. A method according to claim 22, wherein the nucleotide sequence being repaired by homologous recombination in the cell encodes a genetic disease.

24. A method for inhibiting a viral infection in a host cell comprising contacting the host cell with a vector according to claim 15 that targets an infecting or integrated viral genome in the host cell or blocks expression of a cellular viral receptor and thereby prevents viral spread in a host organism.


SEQUENCE LISTING (22 sequences)

SEQ ID NO: 1 - protein, 9 aa, Artificial
  Motif
LAGLIDADG

SEQ ID NO: 2 - DNA, 61 bp, Artificial
  Oligonucleotide K227M
caaaatgcgcctgtcaaattattagggaatatgaaattacaatataaattatggttaaaac

SEQ ID NO: 3 - DNA, 40 bp, Artificial
  Oligonucleotide Q171K
cgctagttttgatattgctaaacgcgatggggatattctg

SEQ ID NO: 4 - DNA, 21 bp, Artificial
  Oligonucleotide primer SP149
cgtaatacgactcactatagg

SEQ ID NO: 5 - DNA, 21 bp, Artificial
  Oligonucleotide primer SP150
cgcaattaatgtgagttagct

SEQ ID NO: 6 - DNA, 37 bp, Artificial
  Oligonucleotide Usrf
gagctcggtaccctcgaggccggacacgctgaacttg

SEQ ID NO: 7 - DNA, 36 bp, Artificial
  Usrr
ctcgagggtaccgagctcacctacggcaagctgacc

SEQ ID NO: 8 - DNA, 27 bp, Artificial
  Anif
tcgatgaggaggtttctctgtaaagct

SEQ ID NO: 9 - DNA, 19 bp, Artificial
  Anir
ttacagagaaacctcctca

SEQ ID NO: 10 - DNA, 36 bp, Artificial
  Lib4f
tcgatgaggaggttactctgttataacagctgagct

SEQ ID NO: 11 - DNA, 30 bp, Artificial
  Lib4r
ggtgaggaggttactctgttatagggataa

SEQ ID NO: 12 - DNA, 30 bp, Artificial; identical to SEQ ID NO: 11
  AniCut.F1

SEQ ID NO: 13 - DNA, 30 bp, Artificial
  AniCut.R1
ccctataacagagtaacctcctcaccttat

SEQ ID NO: 14 - DNA, 45 bp, Artificial
  K227M Substitution
cctgtcaaattgttaggcaacatgaaactgcaatacaagttgtgg

SEQ ID NO: 15 - DNA, 48 bp, Artificial
  Oligonucleotide primer Anid E2F
cgctgatcctttgcttacagagaaacctcctcatacgcccacgcgatg

SEQ ID NO: 16 - DNA, 48 bp, Artificial
  Oligonucleotide primer Anid E2R
catcgcgtgggcgtatgaggaggtttctctgtaagcaaaggatcagcg

SEQ ID NO: 17 - DNA, 44 bp, Artificial
  Oligonucleotide primer pCnZPNOA2
gctgatccttttacagagaaacctcctcatgggtaacagtcttg

SEQ ID NO: 18 - DNA, 44 bp, Artificial
  Oligonucleotide primer pCnZPNOA3
caagactgttacccatgaggaggtttctctgtaaaaggatcagc

SEQ ID NO: 19 - DNA, 25 bp, Artificial
  Oligonucleotide primer nExodusK227M-F
gttaggcaacatgaaactgcaatac

SEQ ID NO: 20 - DNA, 25 bp, Artificial
  Oligonucleotide primer nExodusK227M-R
gtattgcagtttcatgttgcctaac

SEQ ID NO: 21 - DNA, 28 bp, Artificial
  Oligonucleotide primer dExodusE148Q-F
gatttatagaagctcagggctgtttcag

SEQ ID NO: 22 - DNA, 28 bp, Artificial
  Oligonucleotide primer dExodusE148Q-R
ctgaaacagccctgagcttctataaatc